(19) United States
(12) Patent Application Publication
(10) Pub. No.: US 2022/0127255 A1
(43) Pub. Date: Apr. 28, 2022

(54) HERBICIDAL COMPOUNDS

(71) Applicant: SYNGENTA CROP PROTECTION AG, Basel (CH)

(72) Inventors: Nigel James WILLETTS, Bracknell, Berkshire (GB); Andrea MCGRANAGHAN, Bracknell, Berkshire (GB); James Nicholas SCUTT, Bracknell, Berkshire (GB)

(73) Assignee: SYNGENTA CROP PROTECTION AG, Basel (GB)

(21) Appl. No.: 17/435,253

(22) PCT Filed: Mar. 5, 2020
(86) PCT No.: PCT/EP2020/055935

§ 371 Date: Aug. 31, 2021

(30) Foreign Application Priority Data
Mar. 6, 2019 (GB) 1903000.6

(51) Int. Cl. C07D 417/14; A01N 43/82; A01N 43/78; A01N 43/80; C07D 413/14; C07D 403/14; C07D 405/14; A01N 43/713

(52) U.S. Cl. CPC C07D 417/14; A01N 43/82; A01N 43/78; A01N 43/713; C07D 413/14; C07D 403/14; C07D 405/14; A01N 43/80

(57) ABSTRACT

The present invention relates to herbicidally active pyridazine derivatives of Formula (I) as defined herein, as well as to herbicidal compositions comprising such derivatives, and the use of such compounds and compositions for controlling undesirable plant growth: in particular the use for controlling weeds, in crops of useful plants.

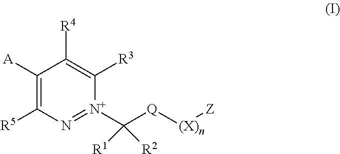

International Patent Application number PCT/EP2018/072280 describes herbicidal N-alkyl pyridazinyl betaine derivatives.

The present invention relates to herbicidally active pyridazine derivatives, as well as to processes and intermediates used for the preparation of such derivatives. The invention further extends to herbicidal compositions comprising such derivatives, as well as to the use of such compounds and compositions for controlling undesirable plant growth: in particular the use for controlling weeds, in crops of useful plants.

The present invention is based on the finding that pyridazine derivatives of Formula (I), wherein Z terminates in a heterocylic or heteroaromatic moiety as defined herein, exhibit surprisingly good herbicidal activity. Thus, according to the present invention there is provided a compound of Formula (I) or an agronomically acceptable salt or zwitterionic species thereof:

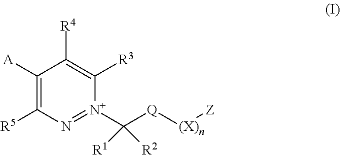

wherein

R¹ is selected from the group consisting of hydrogen, halogen, C₁-C₆alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵;

R² is selected from the group consisting of hydrogen, halogen, C₁-C₆alkyl and C₁-C₆haloalkyl;

and wherein when R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, R² is selected from the group consisting of hydrogen and C₁-C₆alkyl;

or R¹ and R² together with the carbon atom to which they are attached form a C₃-C₆cycloalkyl ring or a 3- to 6-membered heterocyclyl, which comprises 1 or 2 heteroatoms individually selected from N and O;

Q is (CR^{1a}R^{2b})ₘ;

m is 0, 1, 2 or 3;

each R^{1a} and R^{2b} are independently selected from the group consisting of hydrogen, halogen, C₁-C₆alkyl, C₁-C₆haloalkyl, —OH, —OR⁷, —OR^{15a}, —NH₂, —NHR⁷, —NHR^{15a}, —N(R⁶)CHO, —NR^{7b}R^{7c} and —S(O)ᵣR¹⁵;

or each R^{1a} and R^{2b} together with the carbon atom to which they are attached form a C₃-C₆cycloalkyl ring or a 3- to 6-membered heterocyclyl, which comprises 1 or 2 heteroatoms individually selected from N and O;

R³, R⁴ and R⁵ are independently selected from the group consisting of hydrogen, halogen, cyano, nitro, —S(O)ᵣR¹⁵, C₁-C₆alkyl, C₁-C₆fluoroalkyl, C₁-C₆fluoroalkoxy, C₁-C₆alkoxy, C₃-C₆cycloalkyl and —N(R⁶)₂;

each R⁶ is independently selected from hydrogen and C₁-C₆alkyl;

each R⁷ is independently selected from the group consisting of C₁-C₆alkyl, —S(O)₂R¹⁵, —C(O)R¹⁵, —C(O)OR¹⁵ and —C(O)NR¹⁶R¹⁷;

each R^{7a} is independently selected from the group consisting of —S(O)₂R¹⁵, —C(O)R¹⁵, —C(O)OR¹⁵, —C(O)NR¹⁶R¹⁷ and —C(O)NR⁶R^{15a};

R^{7b} and R^{7c} are independently selected from the group consisting of C₁-C₆alkyl, —S(O)₂R¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different;

or R^{7b} and R^{7c} together with the nitrogen atom to which they are attached form a 4- to 6-membered heterocyclyl ring which optionally comprises one additional heteroatom individually selected from N, O and S;

A is a 6-membered heteroaryl, which comprises 1, 2, 3 or 4 nitrogen atoms and wherein the heteroaryl may be optionally substituted by 1, 2, 3 or 4 R⁸ substituents, which may be the same or different;

and wherein when A is substituted by 1 or 2 substituents, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy, C₃-C₆alkynyloxy, N—C₃-C₆cycloalkylamino, —C(R⁶)═NOR⁶, phenyl, a 3- to 6-membered heterocyclyl, which comprises 1 or 2 heteroatoms individually selected from N and O, and a 5- or 6-membered heteroaryl, which comprises 1, 2, 3 or 4 heteroatoms individually selected from N, O and S, and wherein said phenyl, heterocyclyl or heteroaryl are optionally substituted by 1, 2 or 3 R⁹ substituents, which may be the same or different;

and wherein when A is substituted by 3 or 4 substituents, each R⁸ is independently selected from the group consisting of halogen, —NH₂, —NHR⁷, —N(R⁷)₂, —OH, —OR⁷, —C(O)NR¹⁶R¹⁷, —S(O)₂NR¹⁶R¹⁷, C₁-C₆alkyl and C₁-C₆haloalkyl;

each R⁹ is independently selected from the group consisting of halogen, cyano, nitro, C₁-C₄alkyl, C₁-C₄alkoxy, C₁-C₄haloalkyl and C₁-C₄haloalkoxy;

X is selected from the group consisting of C₃-C₆cycloalkyl, phenyl, a 5- or 6-membered heteroaryl, which comprises 1, 2, 3 or 4 heteroatoms individually selected from N, O and S, and a 4- to 6-membered heterocyclyl, which comprises 1, 2 or 3 heteroatoms individually selected from N, O and S, and wherein said cycloalkyl, phenyl, heteroaryl or heterocyclyl moieties are optionally substituted by 1 or 2 R⁹ substituents, and wherein the aforementioned CR¹R², Q and Z moieties may be attached at any position of said cycloalkyl, phenyl, heteroaryl or heterocyclyl moieties;

n is 0 or 1;

Z is an organic acid comprising: a terminal cyclic moiety other than phenyl, at least one oxygen atom, and at least one further heteroatom independently selected from O, N and S;

R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different;

R¹⁵ is selected from the group consisting of C₁-C₆alkyl and phenyl, and wherein said phenyl is optionally substituted by 1, 2 or 3 R⁹ substituents, which may be the same or different;

R^{15a} is phenyl, wherein said phenyl is optionally substituted by 1, 2 or 3 R⁹ substituents, which may be the same or different;

R¹⁶ and R¹⁷ are independently selected from the group consisting of hydrogen and C₁-C₆alkyl; or R¹⁶ and R¹⁷ together with the nitrogen atom to which they are attached form a 4- to 6-membered heterocyclyl ring which optionally comprises one additional heteroatom individually selected from N, O and S; and

r is 0, 1 or 2.

According to a second aspect of the invention, there is provided an agrochemical composition comprising a herbicidally effective amount of a compound of Formula (I) and an agrochemically-acceptable diluent or carrier. Such an agricultural composition may further comprise at least one additional active ingredient.

According to a third aspect of the invention, there is provided a method of controlling or preventing undesirable plant growth, wherein a herbicidally effective amount of a compound of Formula (I), or a composition comprising this compound as active ingredient, is applied to the plants, to parts thereof or the locus thereof.

According to a fourth aspect of the invention, there is provided the use of a compound of Formula (I) as a herbicide.

As used herein, the term “halogen” or “halo” refers to fluorine (fluoro), chlorine (chloro), bromine (bromo) or iodine (iodo), preferably fluorine, chlorine or bromine.

As used herein, cyano means a —CN group.

As used herein, hydroxy means an —OH group.

As used herein, nitro means an —NO₂ group.

As used herein, the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. C₁-C₄alkyl and C₁-C₂alkyl are to be construed accordingly. Examples of C₁-C₆alkyl include, but are not limited to, methyl (Me), ethyl (Et), n-propyl, 1-methylethyl (iso-propyl), n-butyl, and 1-dimethylethyl (t-butyl).

As used herein, the term “C₁-C₆alkoxy” refers to a radical of the formula —ORₐ where Rₐ is a C₁-C₆alkyl radical as generally defined above. C₁-C₄alkoxy is to be construed accordingly. Examples of C₁-C₄alkoxy include, but are not limited to, methoxy, ethoxy, propoxy, iso-propoxy and t-butoxy.

As used herein, the term “C₁-C₆haloalkyl” refers to a C₁-C₆alkyl radical as generally defined above substituted by one or more of the same or different halogen atoms. C₁-C₄haloalkyl is to be construed accordingly. Examples of C₁-C₆haloalkyl include, but are not limited to chloromethyl, fluoromethyl, fluoroethyl, difluoromethyl, trifluoromethyl and 2,2,2-trifluoroethyl.

As used herein, the term “C₂-C₆alkenyl” refers to a straight or branched hydrocarbon chain radical group consisting solely of carbon and hydrogen atoms, containing at least one double bond that can be of either the (E)- or (2)-configuration, having from two to six carbon atoms, which is attached to the rest of the molecule by a single bond. C₂-C₄alkenyl is to be construed accordingly. Examples of C₂-C₆alkenyl include, but are not limited to, prop-1-enyl, allyl (prop-2-enyl) and but-1-enyl.

As used herein, the term “C₂-C₆haloalkenyl” refers to a C₂-C₆alkenyl radical as generally defined above substituted by one or more of the same or different halogen atoms. Examples of C₂-C₆haloalkenyl include, but are not limited to chloroethylene, fluoroethylene, 1,1-difluoroethylene, 1,1-dichloroethylene and 1,1,2-trichloroethylene.

As used herein, the term “C₂-C₆alkynyl” refers to a straight or branched hydrocarbon chain radical group consisting solely of carbon and hydrogen atoms, containing at least one triple bond, having from two to six carbon atoms, and which is attached to the rest of the molecule by a single bond. C₂-C₄alkynyl is to be construed accordingly. Examples of C₂-C₆alkynyl include, but are not limited to, prop-1-ynyl, propargyl (prop-2-ynyl) and but-1-ynyl.

As used herein, the term “C₁-C₆haloalkoxy” refers to a C₁-C₆alkoxy group as defined above substituted by one or more of the same or different halogen atoms. C₁-C₄haloalkoxy is to be construed accordingly. Examples of C₁-C₆haloalkoxy include, but are not limited to, fluoromethoxy, difluoromethoxy, fluoroethoxy, trifluoromethoxy and trifluoroethoxy.

As used herein, the term “C₁-C₃haloalkoxyC₁-C₃alkyl” refers to a radical of the formula R_{b}—O—Rₐ— where R_{b} is a C₁-C₃haloalkyl radical as generally defined above, and Rₐ is a C₁-C₃alkylene radical as generally defined above.

As used herein, the term “C₁-C₃alkoxyC₁-C₃alkyl” refers to a radical of the formula R_{b}—O—Rₐ— where R_{b} is a C₁-C₃alkyl radical as generally defined above, and Rₐ is a C₁-C₃alkylene radical as generally defined above.

As used herein, the term “C₁-C₃alkoxyC₁-C₃alkoxy-” refers to a radical of the formula R_{b}—O—Rₐ—O— where R_{b} is a C₁-C₃alkyl radical as generally defined above, and Rₐ is a C₁-C₃alkylene radical as generally defined above.

As used herein, the term “C₃-C₆alkenyloxy” refers to a radical of the formula —ORₐ where Rₐ is a C₃-C₆alkenyl radical as generally defined above.

As used herein, the term “C₃-C₆alkynyloxy” refers to a radical of the formula —ORₐ where Rₐ is a C₃-C₆alkynyl radical as generally defined above.

As used herein, the term “hydroxyC₁-C₆alkyl” refers to a C₁-C₆alkyl radical as generally defined above substituted by one or more hydroxy groups.

As used herein, the term “C₁-C₆alkylcarbonyl” refers to a radical of the formula —C(O)Rₐ where Rₐ is a C₁-C₆alkyl radical as generally defined above.

As used herein, the term “C₁-C₆alkoxycarbonyl” refers to a radical of the formula —C(O)ORₐ where Rₐ is a C₁-C₆alkyl radical as generally defined above.

As used herein, the term “aminocarbonyl” refers to a radical of the formula —C(O)NH₂.

As used herein, the term “C₃-C₆cycloalkyl” refers to a stable, monocyclic ring radical which is saturated or partially unsaturated and contains 3 to 6 carbon atoms. C₃-C₄cycloalkyl is to be construed accordingly. Examples of C₃-C₆cycloalkyl include, but are not limited to, cyclopropyl, cyclobutyl, cyclopentyl and cyclohexyl.

As used herein, the term “C₃-C₆halocycloalkyl” refers to a C₃-C₆cycloalkyl radical as generally defined above substituted by one or more of the same or different halogen atoms. C₃-C₄halocycloalkyl is to be construed accordingly.

As used herein, the term “C₃-C₆cycloalkoxy” refers to a radical of the formula —ORₐ where Rₐ is a C₃-C₆cycloalkyl radical as generally defined above.

As used herein, the term “N—C₃-C₆cycloalkylamino” refers to a radical of the formula —NHRₐ where Rₐ is a C₃-C₆cycloalkyl radical as generally defined above.

As used herein, except where explicitly stated otherwise, the term “heteroaryl” or “heteroaromatic” refers to a 5- or 6-membered monocyclic aromatic ring which comprises 1, 2, 3 or 4 heteroatoms individually selected from nitrogen, oxygen and sulfur. The heteroaryl radical may be bonded to the rest of the molecule via a carbon atom or heteroatom. Examples of heteroaryl include, furyl, pyrrolyl, imidazolyl, thienyl, pyrazolyl, thiazolyl, isothiazolyl, oxazolyl, isoxazolyl, triazolyl, tetrazolyl, pyrazinyl, pyridazinyl, pyrimidyl or pyridyl.

As used herein, except where explicitly stated otherwise, the term “heterocyclyl” or “heterocyclic” refers to a stable 4- to 6-membered non-aromatic monocyclic ring radical which comprises 1, 2, 3 or 4 heteroatoms individually selected from nitrogen, oxygen and sulfur. The heterocyclyl radical may be bonded to the rest of the molecule via a carbon atom or heteroatom. Examples of heterocyclyl include, but are not limited to, pyrrolinyl, pyrrolidyl, tetrahydrofuryl, tetrahydrothienyl, tetrahydrothiopyranyl, piperidyl, piperazinyl, tetrahydropyranyl, dihydroisoxazolyl, dioxolanyl, morpholinyl or 6-lactamyl.

The presence of one or more possible asymmetric carbon atoms in a compound of Formula (I) means that the compounds may occur in chiral isomeric forms, i.e., enantiomeric or diastereomeric forms. Also atropisomers may occur as a result of restricted rotation about a single bond. Formula (I) is intended to include all those possible isomeric forms and mixtures thereof. The present invention includes all those possible isomeric forms and mixtures thereof for a compound of Formula (I). Likewise, Formula (I) is intended to include all possible tautomers (including lactam-lactim tautomerism and keto-enol tautomerism) where present. The present invention includes all possible tautomeric forms for a compound of Formula (I). Similarly, where there are di-substituted alkenes, these may be present in E or Z form or as mixtures of both in any proportion. The present invention includes all these possible isomeric forms and mixtures thereof for a compound of Formula (I).

The compounds of Formula (I) will typically be provided in the form of an agronomically acceptable salt, a zwitterion or an agronomically acceptable salt of a zwitterion. This invention covers all such agronomically acceptable salts, zwitterions and mixtures thereof in all proportions.

For example a compound of Formula (I) wherein Z comprises an acidic proton, may exist as a zwitterion, a compound of formula (I-I), or as an agronomically acceptable salt, a compound of formula (I-II) as shown below:

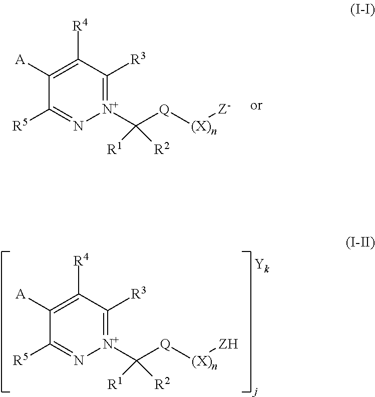

wherein, Y represents an agronomically acceptable anion and j and k represent integers that may be selected from 1, 2 or 3, dependent upon the charge of the respective anion Y.

A compound of Formula (I) may also exist as an agronomically acceptable salt of a zwitterion, a compound of formula (I-III) as shown below:

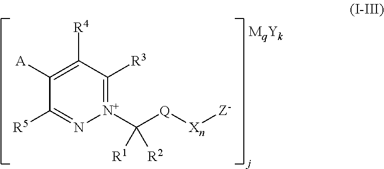

wherein, Y represents an agronomically acceptable anion, M represents an agronomically acceptable cation (in addition to the pyridazinium cation) and the integers j, k and q may be selected from 1, 2 or 3, dependent upon the charge of the respective anion Y and respective cation M.

Thus where a compound of Formula (I) is drawn in protonated form herein, the skilled person would appreciate that it could equally be represented in unprotonated or salt form with one or more relevant counter ions.

In one embodiment of the invention there is provided a compound of formula (I-II) wherein k is 2, j is 1 and Y is selected from the group consisting of halogen, trifluoroacetate and pentafluoropropionate. In this embodiment a nitrogen atom in ring A may be protonated or a nitrogen atom comprised in R¹, R², Q or X may be protonated. Preferably, in a compound of formula (I-II), k is 2, j is 1 and Y is chloride, wherein a nitrogen atom in ring A is protonated.

Suitable agronomically acceptable salts of the present invention, represented by an anion Y, include but are not limited chloride, bromide, iodide, fluoride, 2-naphthalenesulfonate, acetate, adipate, methoxide, ethoxide, propoxide, butoxide, aspartate, benzenesulfonate, benzoate, bicarbonate, bisulfate, bitartrate, butylsulfate, butylsulfonate, butyrate, camphorate, camsylate, caprate, caproate, caprylate, carbonate, citrate, diphosphate, edetate, edisylate, enanthate, ethanedisulfonate, ethanesulfonate, ethylsulfate, formate, fumarate, gluceptate, gluconate, glucoronate, glutamate, glycerophosphate, heptadecanoate, hexadecanoate, hydrogen sulfate, hydroxide, hydroxynaphthoate, isethionate, lactate, lactobionate, laurate, malate, maleate, mandelate, mesylate, methanedisulfonate, methylsulfate, mucate, myristate, napsylate, nitrate, nonadecanoate, octadecanoate, oxalate, pelargonate, pentadecanoate, pentafluoropropionate, perchlorate, phosphate, propionate, propylsulfate, propylsulfonate, succinate, sulfate, tartrate, tosylate, tridecylate, triflate, trifluoroacetate, undecylinate and valerate.

Suitable cations represented by M include, but are not limited to, metals, conjugate acids of amines and organic cations. Examples of suitable metals include aluminium, calcium, cesium, copper, lithium, magnesium, manganese, potassium, sodium, iron and zinc. Examples of suitable amines include allylamine, ammonia, amylamine, arginine, benethamine, benzathine, butenyl-2-amine, butylamine, butylethanolamine, cyclohexylamine, decylamine, diamylamine, dibutylamine, diethanolamine, diethylamine, diethylenetriamine, diheptylamine, dihexylamine, diisoamylamine, diisopropylamine, dimethylamine, dioctylamine, dipropanolamine, dipropargylamine, dipropylamine, dodecylamine, ethanolamine, ethylamine, ethylbutylamine, ethylenediamine, ethylheptylamine, ethyloctylamine, ethylpropanolamine, heptadecylamine, heptylamine, hexadecylamine, hexenyl-2-amine, hexylamine, hexylheptylamine, hexyloctylamine, histidine, indoline, isoamylamine, isobutanolamine, isobutylamine, isopropanolamine, isopropylamine, lysine, meglumine, methoxyethylamine, methylamine, methylbutylamine, methylethylamine, methylhexylamine, methylisopropylamine, methylnonylamine, methyloctadecylamine, methylpentadecylamine, morpholine, N,N-diethylethanolamine, N-methylpiperazine, nonylamine, octadecylamine, octylamine, oleylamine, pentadecylamine, pentenyl-2-amine, phenoxyethylamine, picoline, piperazine, piperidine, propanolamine, propylamine, propylenediamine, pyridine, pyrrolidine, sec-butylamine, stearylamine, tallowamine, tetradecylamine, tributylamine, tridecylamine, trimethylamine, triheptylamine, trihexylamine, triisobutylamine, triisodecylamine, triisopropylamine, trimethylamine, tripentylamine, tripropylamine, tris(hydroxymethyl)aminomethane, and undecylamine. Examples of suitable organic cations include benzyltributylammonium, benzyltrimethylammonium, benzyltriphenylphosphonium, choline, tetrabutylammonium, tetrabutylphosphonium, tetraethylammonium, tetraethylphosphonium, tetramethylammonium, tetramethylphosphonium, tetrapropylammonium, tetrapropylphosphonium, tributylsulfonium, tributylsulfoxonium, triethylsulfonium, triethylsulfoxonium, trimethylsulfonium, trimethylsulfoxonium, tripropylsulfonium and tripropylsulfoxonium.

Preferred compounds of Formula (I), wherein Z comprises an acidic proton, can be represented as either (I-I) or (I-II). For compounds of formula (I-II) emphasis is given to salts when Y is chloride, bromide, iodide, hydroxide, bicarbonate, acetate, pentafluoropropionate, triflate, trifluoroacetate, methylsulfate, tosylate and nitrate, wherein j and k are 1. Preferably, Y is chloride, bromide, iodide, hydroxide, bicarbonate, acetate, trifluoroacetate, methylsulfate, tosylate and nitrate, wherein j and k are 1. For compounds of formula (I-II) emphasis is also given to salts when Y is carbonate and sulfate, wherein j is 2 and k is 1, and when Y is phosphate, wherein j is 3 and k is 1.

Where appropriate compounds of Formula (I) may also be in the form of (and/or be used as) an N-oxide.

Compounds of Formula (I) wherein m is 0 and n is 0 may be represented by a compound of Formula (I-Ia) as shown below:

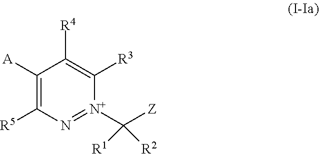

wherein R¹, R², R³, R⁴, R⁵, A and Z are as defined for compounds of Formula (I).

Compounds of Formula (I) wherein m is 1 and n is 0 may be represented by a compound of Formula (I-Ib) as shown below:

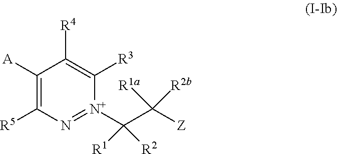

wherein R¹, R², R^{1a}, R^{2b}, R³, R⁴, R⁵, A and Z are as defined for compounds of Formula (I).

Compounds of Formula (I) wherein m is 2 and n is 0 may be represented by a compound of Formula (I-Ic) as shown below:

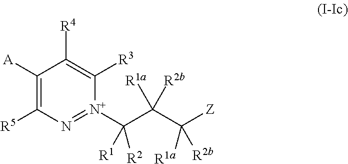

wherein R¹, R², R^{1a}, R^{2b}, R³, R⁴, R⁵, A and Z are as defined for compounds of Formula (I).

Compounds of Formula (I) wherein m is 3 and n is 0 may be represented by a compound of Formula (I-Id) as shown below:

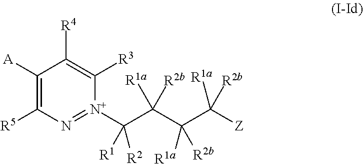

wherein R¹, R², R^{1a}, R^{2b}, R³, R⁴, R⁵, A and Z are as defined for compounds of Formula (I).

The following text provides definitions, including preferred definitions, for substituents n, m, r, A, Q, X, Z, R¹, R², R^{1a}, R^{2b}, R³, R⁴, R⁵, R⁶, R⁷, R^{7a}, R^{7b}, R^{7c}, R⁸, R⁹, R¹⁰, R¹⁵, R^{15a}, R¹⁶, and R¹⁷ with reference to the compounds of Formula (I) according to the invention. For any one of these substituents, any of the definitions given below may be combined with any definition of any other substituent given below or elsewhere in this document.

R¹ is selected from the group consisting of hydrogen, halogen, C₁-C₆alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵. Preferably, R¹ is selected from the group consisting of hydrogen, halogen, C₁-C₆alkyl, C₁-C₆fluoroalkyl, —OR⁷, —NHS(O)₂R¹⁵, —NHC(O)R¹⁵, —NHC(O)OR¹⁵, —NHC(O)NR¹⁶R¹⁷, —N(R^{7a})₂ and —S(O)ᵣR¹⁵. More preferably, R¹ is selected from the group consisting of hydrogen, halogen, C₁-C₆alkyl, C₁-C₆fluoroalkyl, —OR⁷ and —N(R^{7a})₂. Even more preferably, R¹ is selected from the group consisting of hydrogen, C₁-C₆alkyl, —OR⁷ and —N(R^{7a})₂. Even more preferably still, R¹ is hydrogen or C₁-C₆alkyl. Yet even more preferably still, R¹ is hydrogen or methyl. Most preferably R¹ is hydrogen.

R² is selected from the group consisting of hydrogen, halogen, C₁-C₆alkyl and C₁-C₆haloalkyl. Preferably, R² is selected from the group consisting of hydrogen, halogen, C₁-C₆alkyl and C₁-C₆fluoroalkyl. More preferably, R² is hydrogen or C₁-C₆alkyl. Even more preferably, R² is hydrogen or methyl. Most preferably R² is hydrogen. When R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, R² is selected from the group consisting of hydrogen and C₁-C₆alkyl. Preferably, when R¹ is selected from the group consisting of —OR⁷, —NHS(O)₂R¹⁵, —NHC(O)R¹⁵, —NHC(O)OR¹⁵, —NHC(O)NR¹⁶R¹⁷, —N(R^{7a})₂ and —S(O)ᵣR¹⁵, R² is selected from the group consisting of hydrogen and methyl.

Alternatively, R¹ and R² together with the carbon atom to which they are attached form a C₃-C₆cycloalkyl ring or a 3- to 6-membered heterocyclyl, which comprises 1 or 2 heteroatoms individually selected from N and O. Preferably, R¹ and R² together with the carbon atom to which they are attached form a C₃-C₆cycloalkyl ring. More preferably, R¹ and R² together with the carbon atom to which they are attached form a cyclopropyl ring.

In one embodiment R¹ and R² are hydrogen.

In another embodiment R¹ is methyl and R² is hydrogen.

In another embodiment R¹ is methyl and R² is methyl.

Q is (CR^{1a}R^{2b})ₘ, and m is 0, 1, 2 or 3. Preferably, m is 0, 1 or 2. More preferably, m is 1 or 2. Most preferably, m is 1.

Each R^{1a} and R^{2b} are independently selected from the group consisting of hydrogen, halogen, C₁-C₆alkyl, C₁-C₆haloalkyl, —OH, —OR⁷, —OR^{15a}, —NH₂, —NHR⁷, —NHR^{15a}, —N(R⁶)CHO, —NR^{7b}R^{7c} and —S(O)ᵣR¹⁵. Preferably, each R^{1a} and R^{2b} are independently selected from the group consisting of hydrogen, halogen, C₁-C₆alkyl, C₁-C₆fluoroalkyl, —OH, —NH₂ and —NHR⁷. More preferably, each Ria and R^{2b} are independently selected from the group consisting of hydrogen, C₁-C₆alkyl, —OH and —NH₂. Even more preferably, each R^{1a} and R^{2b} are independently selected from the group consisting of hydrogen, methyl, —OH and —NH₂. Even more preferably still, each R^{1a} and R^{2b} are independently selected from the group consisting of hydrogen and methyl. Most preferably R^{1a} and R^{2b} are hydrogen.

In another embodiment each R^{1a} and R^{2b} are independently selected from the group consisting of hydrogen and C₁-C₆alkyl.

Alternatively, each R^{1a} and R^{2b} together with the carbon atom to which they are attached form a C₃-C₆cycloalkyl ring or a 3- to 6-membered heterocyclyl, which comprises 1 or 2 heteroatoms individually selected from N and O. Preferably, each R^{1a} and R^{2b} together with the carbon atom to which they are attached form a C₃-C₆cycloalkyl ring. More preferably, each R^{1a} and R^{2b} together with the carbon atom to which they are attached form a cyclopropyl ring.

R³, R⁴ and R⁵ are each independently selected from the group consisting of hydrogen, halogen, cyano, nitro, —S(O)ᵣR¹⁵, C₁-C₆alkyl, C₁-C₆fluoroalkyl, C₁-C₆fluoroalkoxy, C₁-C₆alkoxy, C₃-C₆cycloalkyl and —N(R⁶)₂. Preferably, R³, R⁴ and R⁵ are independently selected from the group consisting of hydrogen, C₁-C₆alkyl, C₁-C₆fluoroalkyl, C₁-C₆fluoroalkoxy, C₁-C₆alkoxy, C₃-C₆cycloalkyl and —N(R⁶)₂. More preferably, R³, R⁴ and R⁵ are each independently selected from the group consisting of hydrogen, C₁-C₆alkyl and C₁-C₆alkoxy. Even more preferably, R³, R⁴ and R⁵ are each independently selected from the group consisting of hydrogen and C₁-C₆alkyl. Even more preferably still, R³, R⁴ and R⁵ are each independently selected from the group consisting of hydrogen and methyl. Most preferably, R³, R⁴ and R⁵ are hydrogen.

Each R⁶ is independently selected from hydrogen and C₁-C₆alkyl. Preferably, each R⁶ is independently selected from hydrogen and methyl.

Each R⁷ is independently selected from the group consisting of C₁-C₆alkyl, —S(O)₂R¹⁵, —C(O)R¹⁵, —C(O)OR¹⁵ and —C(O)NR¹⁶R¹⁷. Preferably, each R⁷ is independently selected from the group consisting of C₁-C₆alkyl, —C(O)R¹⁵ and —C(O)NR¹⁶R¹⁷. More preferably, each R⁷ is C₁-C₆alkyl. Most preferably, each R⁷ is methyl.

Each R^{7a} is independently selected from the group consisting of —S(O)₂R¹⁵, —C(O)R¹⁵, —C(O)OR¹⁵—C(O)NR¹⁶R¹⁷ and —C(O)NR⁶R^{15a}. Preferably, each R^{7a} is independently —C(O)R¹⁵ or —C(O)NR¹⁶R¹⁷.

R^{7b} and R^{7c} are independently selected from the group consisting of C₁-C₆alkyl, —S(O)₂R¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different. Preferably, R^{7b} and R^{7c} are independently selected from the group consisting of C₁-C₆alkyl, —C(O)R¹⁵ and —C(O)NR¹⁶R¹⁷. More preferably, R^{7b} and R^{7c} are C₁-C₆alkyl. Most preferably, R^{7b} and R^{7c} are methyl.

Alternatively, R^{7b} and R^{7c} together with the nitrogen atom to which they are attached form a 4- to 6-membered heterocyclyl ring which optionally comprises one additional heteroatom individually selected from N, O and S. Preferably, R^{7b} and R^{7c} together with the nitrogen atom to which they are attached form a 5- to 6-membered heterocyclyl ring which optionally comprises one additional heteroatom individually selected from N and O. More preferably, R^{7b} and R^{7c} together with the nitrogen atom to which they are attached form an pyrrolidyl, oxazolidinyl, imidazolidinyl, piperidyl, piperazinyl or morpholinyl group.

A is a 6-membered heteroaryl, which comprises 1, 2, 3 or 4 nitrogen atoms and wherein the heteroaryl may, where feasible, be optionally substituted by 1, 2, 3 or 4 R⁸ substituents, which may be the same or different. Preferably, A is a 6-membered heteroaryl, which comprises 1, 2, 3 or 4 nitrogen atoms and wherein the heteroaryl may, where feasible, be optionally substituted by 1 or 2 R⁸ substituents, which may be the same or different. More preferably, A is a 6-membered heteroaryl, which comprises 1 or 2 nitrogen atoms and wherein the heteroaryl may be optionally substituted by 1 or 2 R⁸ substituents, which may be the same or different.

Further more preferably, A is selected from the group consisting of formula A-I to A-VIII below

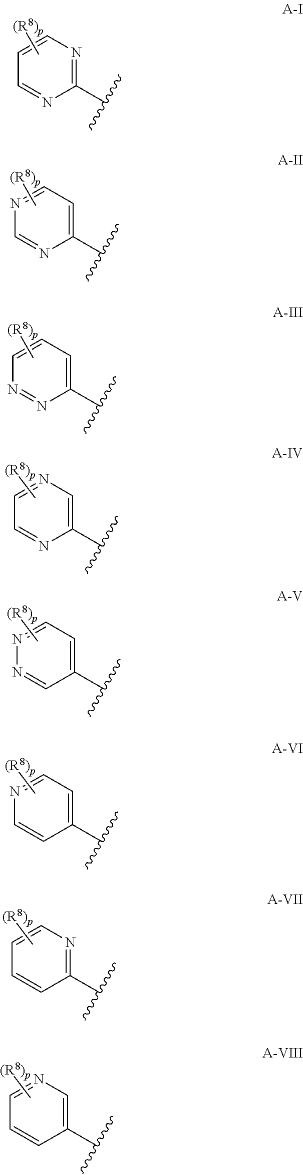

wherein the jagged line defines the point of attachment to the remaining part of a compound of Formula (I) and p is 0, 1 or 2.

Even more preferably, A is selected from the group consisting of formula A-I to A-VII below

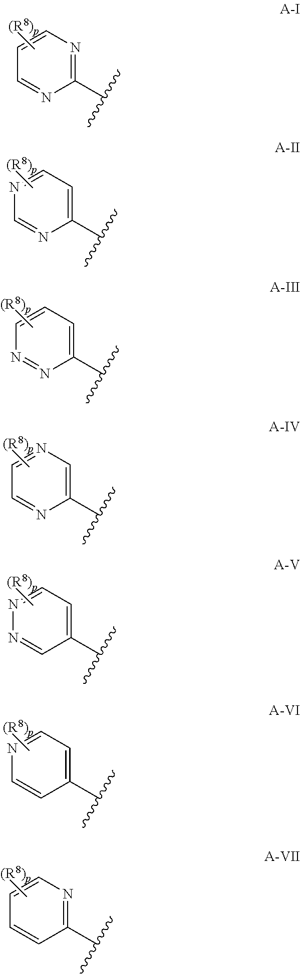

wherein the jagged line defines the point of attachment to the remaining part of a compound of Formula (I) and p is 0, 1 or 2.

Even more preferably still, A is selected from the group consisting of formula A-I to A-V below

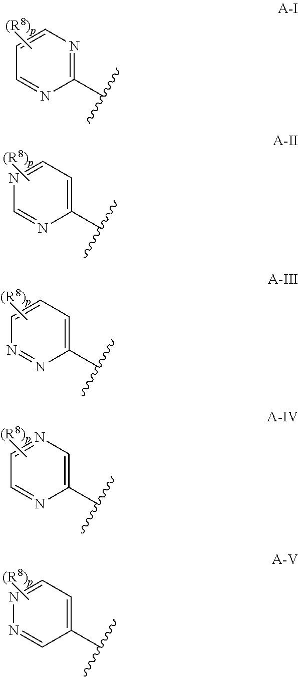

wherein jagged line defines the point of attachment to the remaining part of a compound of Formula (I) and p is 0, 1, or 2.

Yet, even more preferably still, A is selected from the group consisting of formula A-I to A-V and p is 0 or 1. Most preferably, A is selected from the group consisting of formula A-I to A-V and p is 0.

When A is substituted by 1 or 2 substituents each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy, C₃-C₆alkynyloxy, N—C₃-C₆cycloalkylamino, —C(R⁶)═NOR⁶, phenyl, a 3- to 6-membered heterocyclyl, which comprises 1 or 2 heteroatoms individually selected from N and O, and a 5- or 6-membered heteroaryl, which comprises 1, 2, 3 or 4 heteroatoms individually selected from N, O and S, and wherein said phenyl, heterocyclyl or heteroaryl are optionally substituted by 1, 2 or 3 R⁹ substituents, which may be the same or different.

Preferably, when A is substituted by 1 or 2 substituents each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy, C₃-C₆alkynyloxy, —C(R⁶)═NOR⁶, phenyl and a 5- or 6-membered heteroaryl, which comprises 1, 2, 3 or 4 heteroatoms individually selected from N, O and S, and wherein said phenyl or heteroaryl are optionally substituted by 1, 2 or 3 R⁹ substituents, which may be the same or different.

More preferably, when A is substituted by 1 or 2 substituents, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₁-C₃alkoxyC₁-C₃alkyl-, hydroxyC₁-C₆alkyl-, C₁-C₃alkoxyC₁-C₃alkoxy-, C₁-C₆haloalkoxy, phenyl and a 6-membered heteroaryl, which comprises 1 or 2 nitrogen atoms, and wherein said phenyl or heteroaryl are optionally substituted by 1 or 2 R⁹ substituents, which may be the same or different.

Even more preferably, when A is substituted by 1 or 2 substituents, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, hydroxyC₁-C₆alkyl-, C₁-C₆haloalkoxy and a 6-membered heteroaryl, which comprises 1 or 2 nitrogen atoms, and wherein said heteroaryl is optionally substituted by 1 R⁹ substituent.

Even more preferably still, when A is substituted by 1 or 2 substituents, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl.

Further more preferably still, when A is substituted by 1 or 2 substituents, each R⁸ is independently selected from the group consisting of chloro, fluoro, cyano, —NH₂, —N(Me)₂, —OH, —OMe, —S(O)₂Me, —C(O)OMe, —C(O)OH, —C(O)Me, —C(O)NH₂, —C(O)NHMe, —C(O)N(Me)₂, methyl and trifluoromethyl.

Most preferably, when A is substituted by 1 or 2 substituents, each R⁸ is independently selected from the group consisting of chloro, fluoro, cyano, —NH₂, —N(Me)₂, —OMe, —S(O)₂Me, —C(O)NHMe, —C(O)N(Me)₂, methyl and trifluoromethyl.

In one embodiment, when A is substituted by 1 or 2 substituents, each R⁸ is independently selected from the group consisting of halogen, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, hydroxyC₁-C₆alkyl-, and a 6-membered heteroaryl, which comprises 2 nitrogen atoms, and wherein said heteroaryl is optionally substituted by 1 R⁹ substituent. Preferably, when A is substituted by 1 or 2 substituents, each R⁸ is independently selected from the group consisting of chloro, fluoro, cyano, —NH₂, —N(Me)₂, —OH, —OMe, —S(O)₂Me, —C(O)OMe, —C(O)OH, —C(O)Me, —C(O)NH₂, —C(O)NHMe, —C(O)N(Me)₂, —S(O)₂NHMe, methyl, trifluoromethyl, cyclopropyl, hydroxymethyl- and 6-chloropyridazin-3-yl.

Alternatively when A is substituted by 3 or 4 substituents, each R⁸ is independently selected from the group consisting of halogen, —NH₂, —NHR⁷, —N(R⁷)₂, —OH, —OR⁷, —C(O)NR¹⁶R¹⁷, —S(O)₂NR¹⁶R¹⁷, C₁-C₆alkyl and C₁-C₆haloalkyl. Preferably, each R⁸ is independently selected from the group consisting of —NH₂, —NHR⁷, —N(R⁷)₂, —OH, —OR⁷, C₁-C₆alkyl and C₁-C₆haloalkyl. More preferably, each R⁸ is independently selected from the group consisting of —NH₂, —NHR⁷, —OR⁷, C₁-C₆alkyl and C₁-C₆haloalkyl. Even more preferably still, each R⁸ is independently selected from the group consisting of C₁-C₆alkyl and C₁-C₆haloalkyl.

Each R⁹ is independently selected from the group consisting of halogen, cyano, nitro, C₁-C₄alkyl, C₁-C₄alkoxy, C₁-C₄haloalkyl and C₁-C₄haloalkoxy. Preferably, each R⁹ is independently selected from the group consisting of halogen, cyano, C₁-C₄alkyl, C₁-C₄alkoxy, C₁-C₄haloalkyl and C₁-C₄haloalkoxy. More preferably, each R⁹ is independently selected from the group consisting of halogen, C₁-C₄alkyl, C₁-C₄alkoxy and C₁-C₄haloalkyl. Even more preferably, each R⁹ is independently selected from the group consisting of halogen and C₁-C₄alkyl.

X is selected from the group consisting of C₃-C₆cycloalkyl, phenyl, a 5- or 6-membered heteroaryl, which comprises 1, 2, 3 or 4 heteroatoms individually selected from N, O and S, and a 4- to 6-membered heterocyclyl, which comprises 1, 2 or 3 heteroatoms individually selected from N, O and S, and wherein said cycloalkyl, phenyl, heteroaryl or heterocyclyl moieties are optionally substituted by 1 or 2 substituents, which may be the same or different, selected from R⁹, and wherein the aforementioned CR¹R², Q and Z moieties may be attached at any position of said cycloalkyl, phenyl, heteroaryl or heterocyclyl moieties.

Preferably, X is selected from the group consisting of phenyl and a 4- to 6-membered heterocyclyl, which comprises 1 or 2 heteroatoms individually selected from N and O, and wherein said phenyl or heterocyclyl moieties are optionally substituted by 1 or 2 substituents, which may be the same or different, selected from R⁹, and wherein the aforementioned CR¹R², Q and Z moieties may be attached at any position of said phenyl or heterocyclyl moieties.

More preferably, X is a 4- to 6-membered heterocyclyl, which comprises 1 or 2 heteroatoms individually selected from N and O, and wherein said heterocyclyl moieties is optionally substituted by 1 or 2 substituents, which may be the same or different, selected from R⁹, and wherein the aforementioned CR¹R², Q and Z moieties may be attached at any position of said heterocyclyl moiety.

In one embodiment, X is a 5-membered heterocyclyl, which comprises 1 heteroatom, wherein said heteroatom is N, and wherein the aforementioned CR¹R², Q and Z moieties may be attached at any position of said heterocyclyl moiety. Preferably, X is a 5-membered heterocyclyl, which comprises 1 heteroatom, wherein said heteroatom is N, and wherein the aforementioned CR¹R² and Q moieties are attached adjacent to the N atom and the Z moiety is attached to the N atom.

In another embodiment, X is phenyl optionally substituted by 1 or 2 substituents, which may be the same or different, selected from R⁹, and wherein the aforementioned CR¹R², Q and Z moieties may be attached at any position of said phenyl moiety. Preferably, X is phenyl and the aforementioned CR¹R² and Q moieties are attached in a position para to the Z moiety.

As stated herein, n is 0 or 1. Preferably, n is 0.

Z is defined herein as an organic acid comprising a terminal cyclic moiety other than phenyl, at least one oxygen atom, and at least one further heteroatom independently selected from O, N and S. The at least one oxygen atom may be within the cyclic moiety or it may be a substituent elsewhere on Z. When it is a substituent, it may be comprised within that part of Z that attaches it to the rest of the molecule, or it may be a substituent elsewhere on the terminal cyclic moiety. As with the at least one oxygen atom, the at least one further heteroatom may be within the cyclic moiety or it may be a substituent elsewhere on Z. When it is a substituent, it may be comprised within that part of Z that attaches it to the rest of the molecule, or it may be a substituent elsewhere on the terminal cyclic moiety. The skilled man will thus appreciate that Z cannot equal tetrazole per se, or encompass an optionally substituted phenyl or benzyl group by virtue of the proviso.

Z is an acidic moiety, and as such permits compounds of Formula (I) to be represented as a compound of Formula (I-I), a compound of Formula (I-II), or a compound of Formula (I-III) as described above. In general, Z achieves this requirement either by having an —NH— group within its cyclic moiety, through having its cyclic moiety substituted with —OH, or through having an —NH— group elsewhere in Z that is adjacent to a —C(O), a —S(O) or a —S(O)₂ group. In various embodiments Z may have only one, or it may have any two, or it may have all three of these features. Thus in one set of embodiments, a compound of Formula (I) will have a Z group, wherein the cyclic moiety of said Z group (i) is substituted by —OH, and/or (ii) comprises a ring nitrogen bearing a hydrogen atom. In a further set of embodiments a the Z group in a compound of Formula (I) will comprise an —NH— group adjacent to —C(O), —S(O) or —S(O)₂ group.

Thus in a preferred set of embodiments, the terminal cyclic moiety of Z is selected from the group consisting of: an optionally substituted 4-6 membered carbocyclic 1,3-dione; a 5- or 6-membered heterocyclic ring comprising up to 4 heteroatoms independently selected from O, S, and N, substituted either on at least one ring carbon by —OH and/or an a ring nitrogen by hydrogen, and optionally substituted on any other ring atom; a 5- or 6-membered heteroaromatic ring comprising up to 4 heteroatoms independently selected from O, S, and N, substituted either on at least one ring carbon by —OH and/or an a ring nitrogen by hydrogen, and optionally substituted on any other ring atom; and an optionally substituted 5- or 6-membered heteroaromatic ring comprising at least one nitrogen atom and from 0 to 3 further heteroatoms independently selected from O, N and S. Preferably said optional substituents for the 4-6 membered carbocyclic 1,3-dione, the 5- and 6-membered heterocyclic and the 5- and 6-membered heteroaromatic moieties recited in this paragraph will be selected as appropriate from oxo, C₁-C₆alkyl, C₁-C₆alkoxy, C₁-C₃alkoxyC₁-C₃alkyl. In a further set of preferred embodiments, Z will comprise a terminal cyclic moiety other than phenyl, linked to the rest of the compound of Formula (I) via a moiety comprising an —NH— group adjacent to an oxygen containing group selected from the group consisting of —C(O), —S(O), and —S(O)₂.

In a particularly preferred set of embodiments, Z is selected from the group consisting of Z1 to Z69 as shown below:

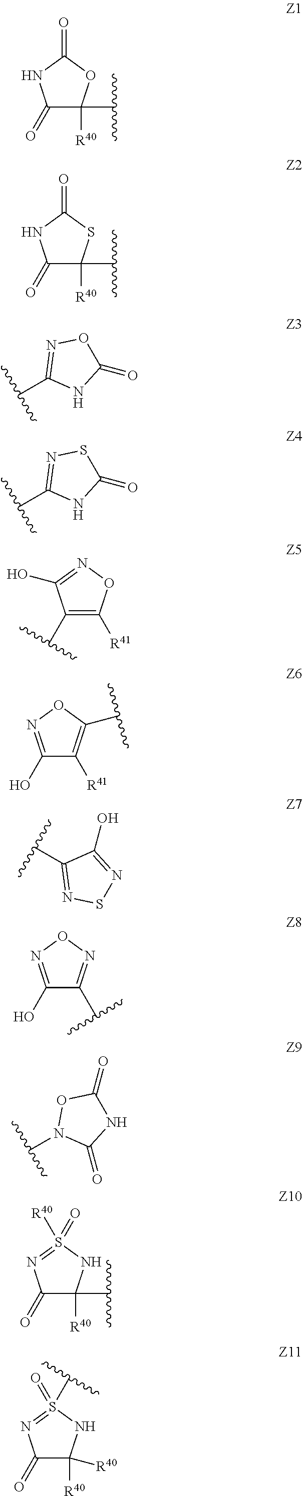

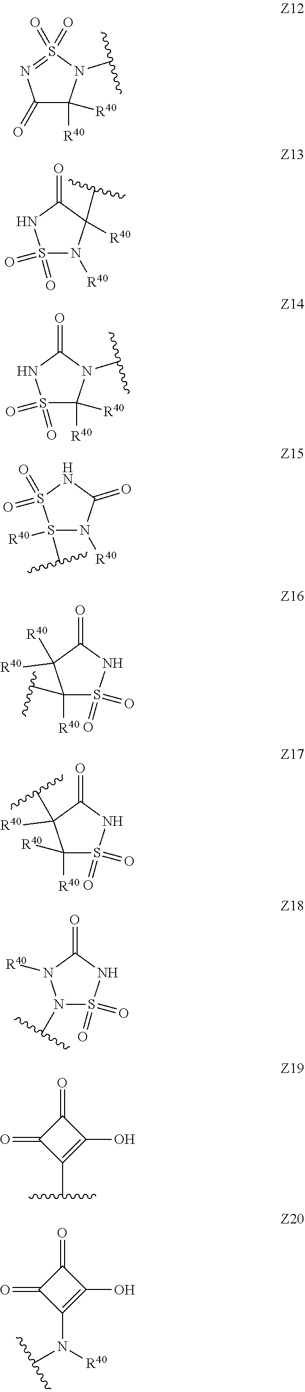

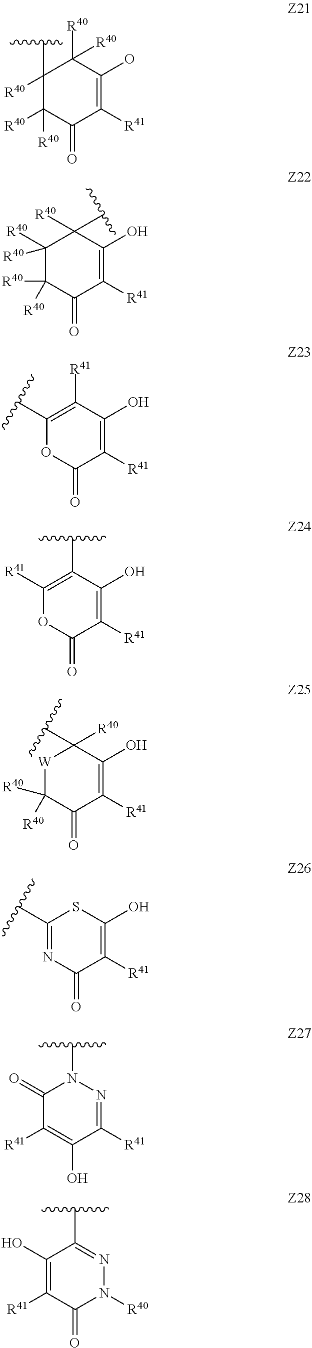

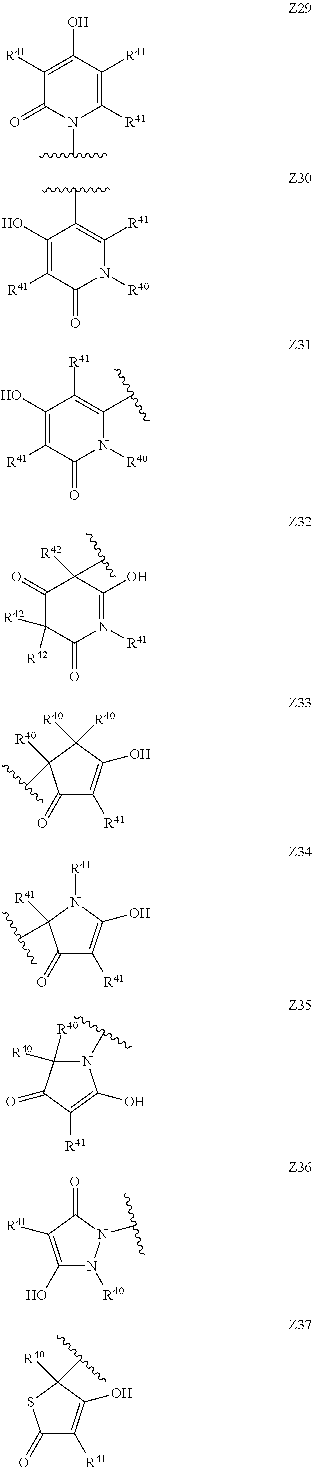

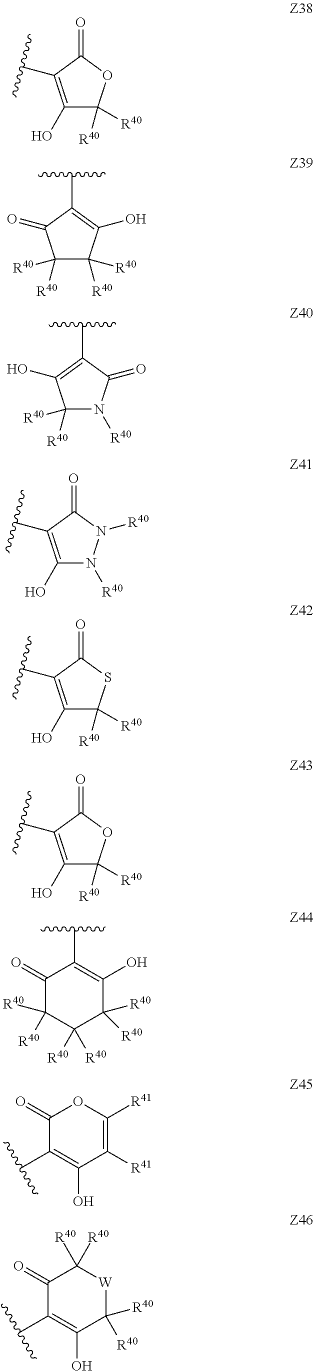

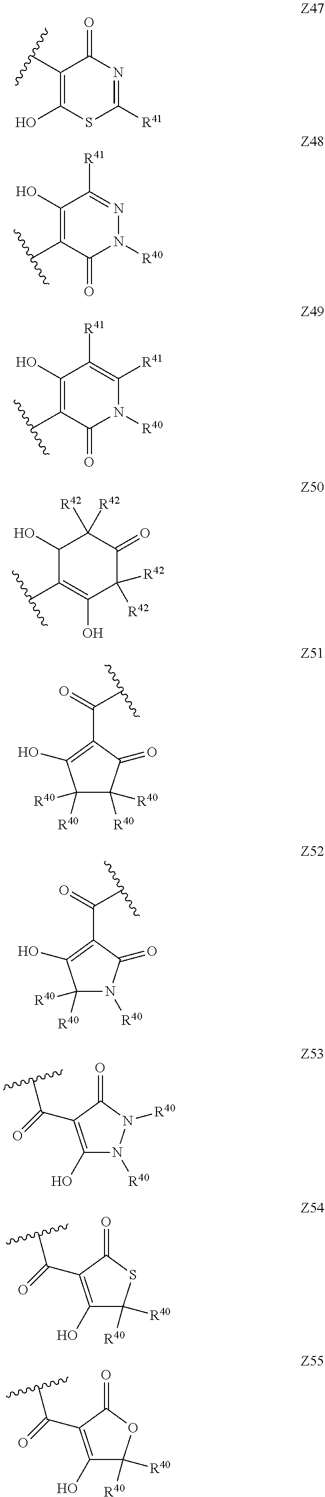

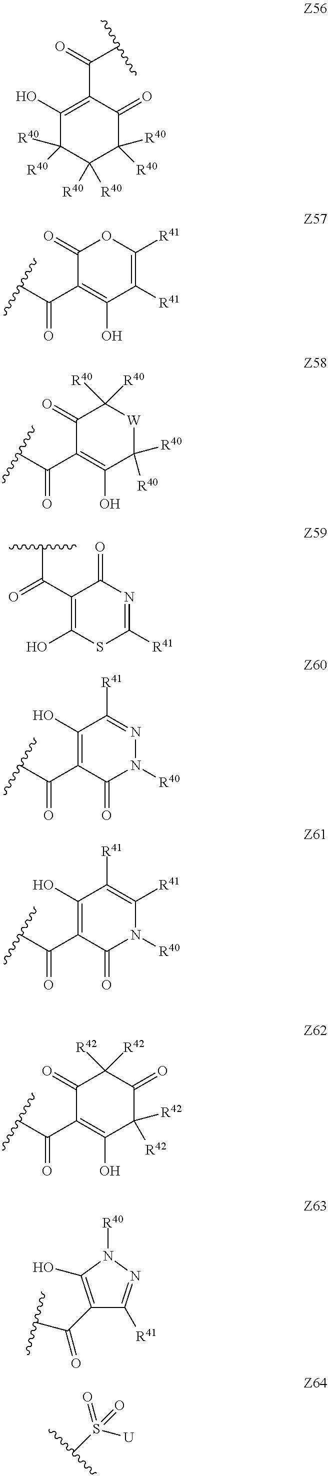

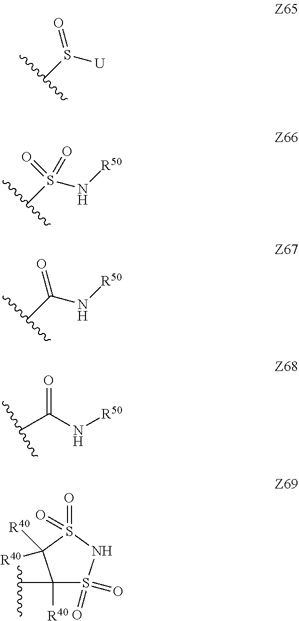

wherein the jagged line denotes the point of attachment to the rest of the molecule and

each R⁴⁰ is independently selected from the group consisting of hydrogen, C₁-C₆alkyl, C₁-C₆alkoxy, C₁-C₃alkoxyC₁-C₃alkyl;

each R⁴¹ is independently selected from the group consisting of hydrogen, C₁-C₆alkyl, and C₁-C₃alkoxyC₁-C₃alkyl;

each R⁴² is independently selected from the group consisting of C₁-C₆alkyl and C₁-C₃alkoxyC₁-C₃alkyl;

W is selected from the group consisting of —O—, —S—, —S(O)—, —S(O)₂— and —N(R⁴⁵)—;

R⁴⁵ is selected from the group consisting of hydrogen, C₁-C₃alkyl, C₁-C₃alkoxy, C₁-C₃alkoxyC₁-C₃alkyl, and C₁-C₆alkylcarbonyl;

U is a 5- or 6-membered heteroaromatic ring comprising at least one ring nitrogen atom substituted by hydrogen, and from 0 to 3 further heteroatoms independently selected from O, N and S, wherein said heteroaromatic ring is optionally substituted by 1, 2, or 3 R⁹ substituents, and

R⁵⁰ is a 5- or 6-membered heteroaromatic ring comprising at least one ring nitrogen atom and from 0 to 3 further heteroatoms independently selected from O, N and S, wherein said heteroaromatic ring is optionally substituted by 1, 2, or 3 R⁹ substituents.

As defined herein, R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different. Preferably, R¹⁰ is selected from the group consisting of hydrogen, C₁-C₆alkyl, phenyl and benzyl. More preferably, R¹⁰ is selected from the group consisting of hydrogen and C₁-C₆alkyl. Most preferably, R¹⁰ is hydrogen.

R¹⁵ is selected from the group consisting of C₁-C₆alkyl and phenyl, and wherein said phenyl is optionally substituted by 1, 2 or 3 R⁹ substituents, which may be the same or different. Preferably, R¹⁵ is selected from the group consisting of C₁-C₆alkyl and phenyl. More preferably, R¹⁵ is C₁-C₆alkyl. Most preferably R¹⁵ is methyl.

R^{15a} is phenyl, wherein said phenyl is optionally substituted by 1, 2 or 3 R⁹ substituents, which may be the same or different. Preferably, R^{15a} is phenyl optionally substituted by 1 R⁹ substituent. More preferably, R^{15a} is phenyl.

R¹⁶ and R¹⁷ are independently selected from the group consisting of hydrogen and C₁-C₆alkyl. Preferably, R¹⁶ and R¹⁷ are independently selected from the group consisting of hydrogen and methyl.

Alternatively, R¹⁶ and R¹⁷ together with the nitrogen atom to which they are attached form a 4- to 6-membered heterocyclyl ring which optionally comprises one additional heteroatom individually selected from N, O and S. Preferably, R¹⁶ and R¹⁷ together with the nitrogen atom to which they are attached form a 5- to 6-membered heterocyclyl ring which optionally comprises one additional heteroatom individually selected from N and O. More preferably, R¹⁶ and R¹⁷ together with the nitrogen atom to which they are attached form an pyrrolidyl, oxazolidinyl, imidazolidinyl, piperidyl, piperazinyl or morpholinyl group.

As stated herein, r is 0, 1 or 2. Preferably, r is 0 or 2.

The compounds in Tables 1 to 27 below illustrate the compounds of the invention. The skilled person would understand that the compounds of Formula (I) may exist as an agronomically acceptable salt, a zwitterion or an agronomically acceptable salt of a zwitterion as described hereinbefore.

TABLE 1
This table discloses 92 specific compounds of the formula (T-1)

| | |
|---|---|
| | (T-1) |
| 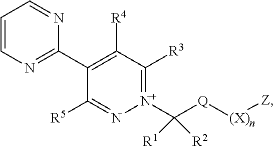 | |

wherein R³, R⁴, R⁵and Z are
as defined in the table below, R¹ and
R² are hydrogen, Q is (CR^{1a}R^{2b})ₘ, m is 0 and n is 0.

| Com- | | | | |
|---|---|---|---|---|
| pound | | | | |
| number | R³ | R⁴ | R⁵ | Z |
| 1.001 | H | H | H | 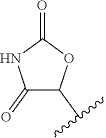 [SMILES: *C1OC(=O)NC1=O] |
| 1.002 | H | H | H | 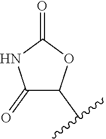 [SMILES: *C1OC(=O)NC1=O] |
| 1.003 | H | H | H | 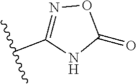 [SMILES: *c1noc(=O)[nH]1] |
| 1.004 | H | H | H | 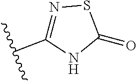 [SMILES: *c1nsc(=O)[nH]1] |
| 1.005 | H | H | H | 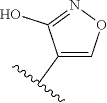 [SMILES: *c1conc1O] |
| 1.006 | H | H | H | 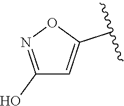 [SMILES: *c1cc(O)no1] |
| 1.007 | H | H | H | 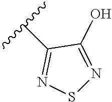 [SMILES: *c1nsnc1O] |
| 1.008 | H | H | H | 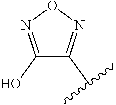 [SMILES: *c1nonc1O] |
| 1.009 | H | H | H | 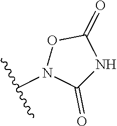 [SMILES: *n1oc(=O)[nH]c1=O] |
| 1.010 | H | H | H | 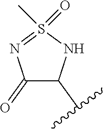 [SMILES: *C1NS(C)(=O)=NC1=O] |
| 1.011 | H | H | H | 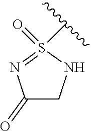 [SMILES: *S1(=O)=NC(=O)CN1] |
| 1.012 | H | H | H | 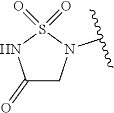 [SMILES: *N1CC(=O)NS1(=O)=O] |
| 1.013 | H | H | H | 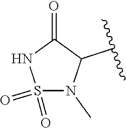 [SMILES: *C1C(=O)NS(=O)(=O)N1C] |
| 1.014 | H | H | H | 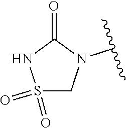 [SMILES: *N1CS(=O)(=O)NC1=O] |
| 1.015 | H | H | H | 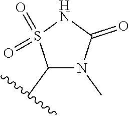 [SMILES: *C1N(C)C(=O)NS1(=O)=O] |
| 1.016 | H | H | H | 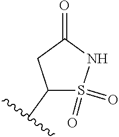 [SMILES: *C1CC(=O)NS1(=O)=O] |
| 1.017 | H | H | H | 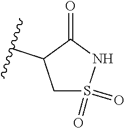 [SMILES: *C1CS(=O)(=O)NC1=O] |
| 1.018 | H | H | H | 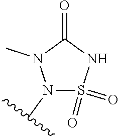 [SMILES: *N1N(C)C(=O)NS1(=O)=O] |
| 1.019 | H | H | H | 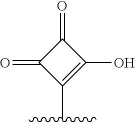 [SMILES: *c1c(O)c(=O)c1=O] |
| 1.020 | H | H | H | 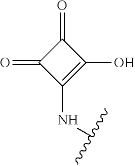 [SMILES: *Nc1c(O)c(=O)c1=O] |
| 1.021 | H | H | H | 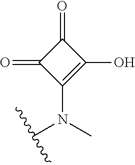 [SMILES: *N(C)c1c(O)c(=O)c1=O] |
| 1.022 | H | H | H | 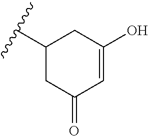 [SMILES: *C1CC(=O)C=C(O)C1] |
| 1.023 | H | H | H | 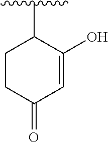 [SMILES: *C1CCC(=O)C=C1O] |
| 1.024 | H | H | H | 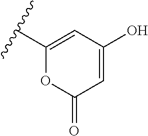 [SMILES: *c1cc(O)cc(=O)o1] |
| 1.025 | H | H | H | 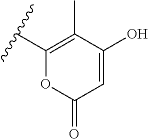 [SMILES: *c1oc(=O)cc(O)c1C] |
| 1.026 | H | H | H | 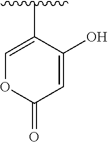 [SMILES: *c1coc(=O)cc1O] |
| 1.027 | H | H | H | 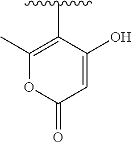 [SMILES: *c1c(O)cc(=O)oc1C] |
| 1.028 | H | H | H | 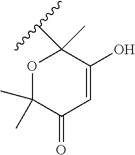 [SMILES: *C1(C)OC(C)(C)C(=O)C=C1O] |
| 1.029 | H | H | H | 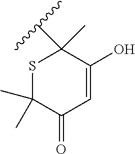 [SMILES: *C1(C)SC(C)(C)C(=O)C=C1O] |
| 1.030 | H | H | H | 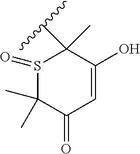 [SMILES: *C1(C)C(O)=CC(=O)C(C)(C)S1=O] |
| 1.031 | H | H | H | 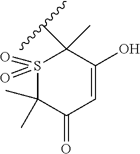 [SMILES: *C1(C)C(O)=CC(=O)C(C)(C)S1(=O)=O] |
| 1.032 | H | H | H | 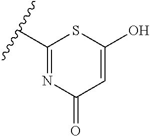 [SMILES: *c1nc(=O)cc(O)s1] |
| 1.033 | H | H | H | 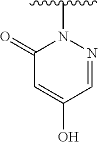 [SMILES: *n1ncc(O)cc1=O] |
| 1.034 | H | H | H | 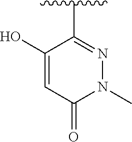 [SMILES: *c1nn(C)c(=O)cc1O] |
| 1.035 | H | H | H | 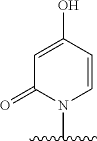 [SMILES: *n1ccc(O)cc1=O] |
| 1.036 | H | H | H | 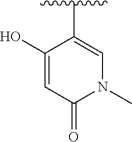 [SMILES: *c1cn(C)c(=O)cc1O] |
| 1.037 | H | H | H | 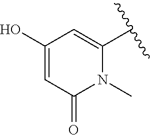 [SMILES: *c1cc(O)cc(=O)n1C] |
| 1.038 | H | H | H | 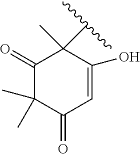 [SMILES: *C1(C)C(=O)C(C)(C)C(=O)C=C1O] |
| 1.039 | H | H | H | 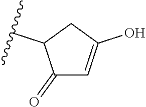 [SMILES: *C1CC(O)=CC1=O] |
| 1.040 | H | H | H | 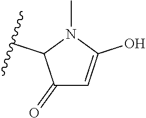 [SMILES: *C1C(=O)C=C(O)N1C] |
| 1.041 | H | H | H | 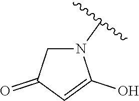 [SMILES: *N1CC(=O)C=C1O] |
| 1.042 | H | H | H | 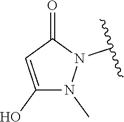 [SMILES: *n1c(=O)cc(O)n1C] |
| 1.043 | H | H | H | 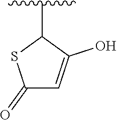 [SMILES: *C1SC(=O)C=C1O] |
| 1.044 | H | H | H | 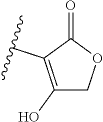 [SMILES: *C1=C(O)COC1=O] |
| 1.045 | H | H | H | 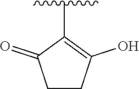 [SMILES: *C1=C(O)CCC1=O] |
| 1.046 | H | H | H | 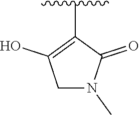 [SMILES: *C1=C(O)CN(C)C1=O] |
| 1.047 | H | H | H | 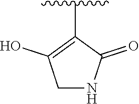 [SMILES: *C1=C(O)CNC1=O] |
| 1.048 | H | H | H | 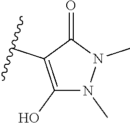 [SMILES: *c1c(O)n(C)n(C)c1=O] |
| 1.049 | H | H | H | 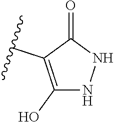 [SMILES: *c1c(O)[nH][nH]c1=O] |
| 1.050 | H | H | H | 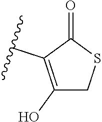 [SMILES: *C1=C(O)CSC1=O] |
| 1.051 | H | H | H | 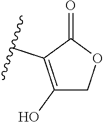 [SMILES: *C1=C(O)COC1=O] |
| 1.052 | H | H | H | 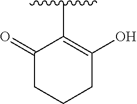 [SMILES: *C1=C(O)CCCC1=O] |
| 1.053 | H | H | H | 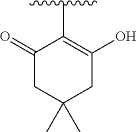 [SMILES: *C1=C(O)CC(C)(C)CC1=O] |
| 1.054 | H | H | H | 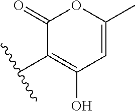 [SMILES: *c1c(O)cc(C)oc1=O] |
| 1.055 | H | H | H | 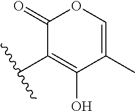 [SMILES: *c1c(O)c(C)coc1=O] |
| 1.056 | H | H | H | 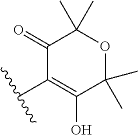 [SMILES: *C1=C(O)C(C)(C)OC(C)(C)C1=O] |
| 1.057 | H | H | H | 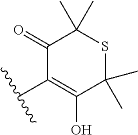 [SMILES: *C1=C(O)C(C)(C)SC(C)(C)C1=O] |
| 1.058 | H | H | H | 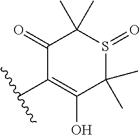 [SMILES: *C1=C(O)C(C)(C)S(=O)C(C)(C)C1=O] |
| 1.059 | H | H | H | 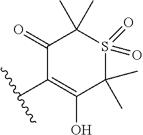 [SMILES: *C1=C(O)C(C)(C)S(=O)(=O)C(C)(C)C1=O] |
| 1.060 | H | H | H | 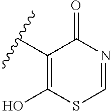 [SMILES: *c1c(O)scnc1=O] |
| 1.061 | H | H | H | 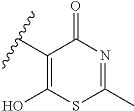 [SMILES: *c1c(O)sc(C)nc1=O] |
| 1.062 | H | H | H | 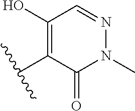 [SMILES: *c1c(O)cnn(C)c1=O] |
| 1.063 | H | H | H | 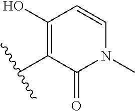 [SMILES: *c1c(O)ccn(C)c1=O] |
| 1.064 | H | H | H | 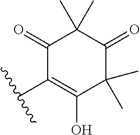 [SMILES: *C1=C(O)C(C)(C)C(=O)C(C)(C)C1=O] |
| 1.065 | H | H | H | 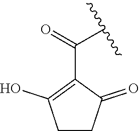 [SMILES: *C(=O)C1=C(O)CCC1=O] |
| 1.066 | H | H | H | 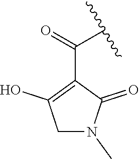 [SMILES: *C(=O)C1=C(O)CN(C)C1=O] |
| 1.067 | H | H | H | 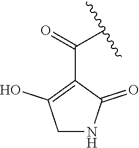 [SMILES: *C(=O)C1=C(O)CNC1=O] |
| 1.068 | H | H | H | 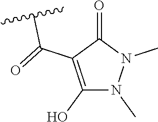 [SMILES: *C(=O)c1c(O)n(C)n(C)c1=O] |
| 1.069 | H | H | H | 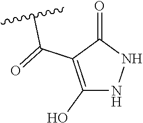 [SMILES: *C(=O)c1c(O)[nH][nH]c1=O] |
| 1.070 | H | H | H | 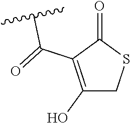 [SMILES: *C(=O)C1=C(O)CSC1=O] |
| 1.071 | H | H | H | 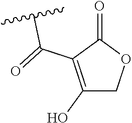 [SMILES: *C(=O)C1=C(O)COC1=O] |
| 1.072 | H | H | H | 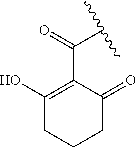 [SMILES: *C(=O)C1=C(O)CCCC1=O] |
| 1.073 | H | H | H | 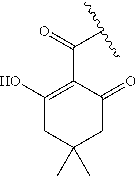 [SMILES: *C(=O)C1=C(O)CC(C)(C)CC1=O] |
| 1.074 | H | H | H | 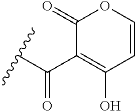 [SMILES: *C(=O)c1c(O)ccoc1=O] |
| 1.075 | H | H | H | 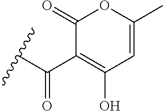 [SMILES: *C(=O)c1c(O)cc(C)oc1=O] |
| 1.076 | H | H | H | 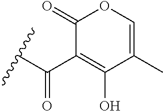 [SMILES: *C(=O)c1c(O)c(C)coc1=O] |
| 1.077 | H | H | H | 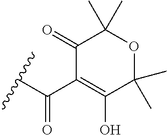 [SMILES: *C(=O)C1=C(O)C(C)(C)OC(C)(C)C1=O] |
| 1.078 | H | H | H | 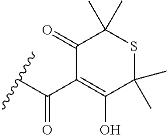 [SMILES: *C(=O)C1=C(O)C(C)(C)SC(C)(C)C1=O] |
| 1.079 | H | H | H | 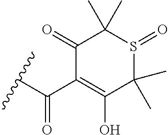 [SMILES: *C(=O)C1=C(O)C(C)(C)S(=O)C(C)(C)C1=O] |
| 1.080 | H | H | H | 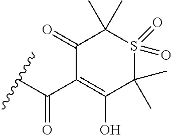 [SMILES: *C(=O)C1=C(O)C(C)(C)S(=O)(=O)C(C)(C)C1=O] |
| 1.081 | H | H | H | 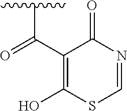 [SMILES: *C(=O)c1c(O)scnc1=O] |
| 1.082 | H | H | H | 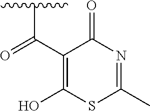 [SMILES: *C(=O)c1c(O)sc(C)nc1=O] |
| 1.083 | H | H | H | 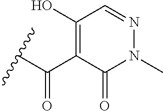 [SMILES: *C(=O)c1c(O)cnn(C)c1=O] |
| 1.084 | H | H | H | 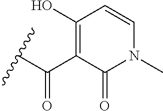 [SMILES: *C(=O)c1c(O)ccn(C)c1=O] |
| 1.085 | H | H | H | 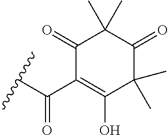 [SMILES: *C(=O)C1=C(O)C(C)(C)C(=O)C(C)(C)C1=O] |
| 1.086 | H | H | H | 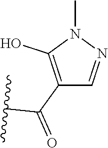 [SMILES: *C(=O)c1cnn(C)c1O] |
| 1.087 | H | H | H | 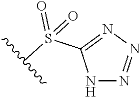 [SMILES: *S(=O)(=O)c1nnn[nH]1] |
| 1.088 | H | H | H | 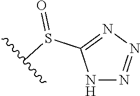 [SMILES: *S(=O)c1nnn[nH]1] |
| 1.089 | H | H | H | 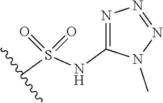 [SMILES: *S(=O)(=O)Nc1nnnn1C] |
| 1.090 | H | H | H | 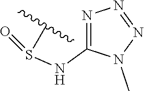 [SMILES: *S(=O)Nc1nnnn1C] |
| 1.091 | H | H | H | 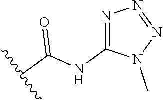 [SMILES: *C(=O)Nc1nnnn1C] |
| 1.092 | H | H | H | 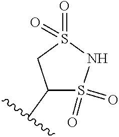 [SMILES: *C1CS(=O)(=O)NS1(=O)=O] |

TABLE 2
This table discloses 92 specific compounds of the formula (T-2)

| | |
|---|---|
| | (T-2) |
| 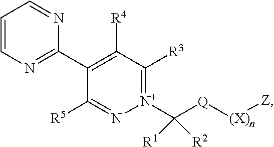 | |

wherein R³, R⁴, R⁵ and Z are as defined in Table 1, R¹ and R² are
hydrogen, Q is CH₂, and n is 0.

TABLE 3
This table discloses 92 specific compounds of the formula (T-3)

| | |
|---|---|
| | (T-3) |
| 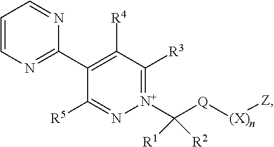 | |

wherein R³, R⁴, R⁵ and Z are as defined in Table 1, R¹ and R² are
hydrogen, Q is CH₂, and n is 0.

TABLE 4
This table discloses 92 specific compounds of the formula (T-4)

| | |
|---|---|
| | (T-4) |
| 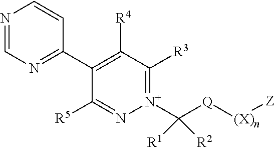 | |

wherein R³, R⁴, R⁵ and Z are as defined in Table 1, R¹ and R² are
hydrogen, Q is (CR^{1a}R^{2b})ₘ, m is 0 and n is 0.

TABLE 5
This table discloses 92 specific compounds of the formula (T-5)

| | |
|---|---|
| | (T-5) |
| 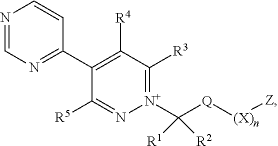 | |

wherein R³, R⁴, R⁵ and Z are as defined in Table 1, R¹ and R² are
hydrogen, Q is CH₂, m is 1 and n is 0.

TABLE 6
This table discloses 92 specific compounds of the formula (T-6)

| | |
|---|---|
| | (T-6) |
| 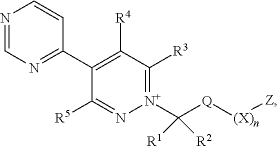 | |

wherein R³, R⁴, R⁵ and Z are as defined in Table 1, R¹ and R² are
hydrogen, Q is CH₂, m is 2 and n is 0.

TABLE 7
This table discloses 92 specific compounds of the formula (T-7)

| | |
|---|---|
| | (T-7) |
| 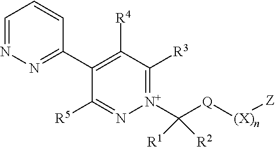 | |

wherein R³, R⁴, R⁵ and Z are as defined in Table 1, R¹ and R² are
hydrogen, Q is (CR^{1a}R^{2b})ₘ, m is 0 and n is 0.

TABLE 8
This table discloses 92 specific compounds of the formula (T-8)

| | |
|---|---|
| | (T-8) |
| 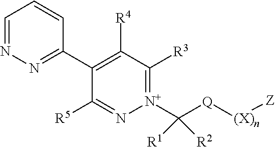 | |

wherein R³, R⁴, R⁵ and Z are as defined in Table 1, R¹ and R² are
hydrogen, Q is CH₂, m is 1 and n is 0.

TABLE 9
This table discloses 92 specific compounds of the formula (T-9)

| | |
|---|---|
| | (T-9) |
| 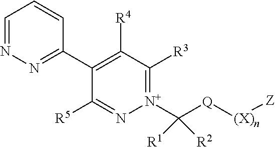 | |

wherein R³, R⁴, R⁵ and Z are as defined in Table 1, R¹ and R² are
hydrogen, Q is CH₂, m is 2 and n is 0.

TABLE 10
This table discloses 92 specific compounds of the formula (T-10)

| | |
|---|---|
| | (T-10) |
| 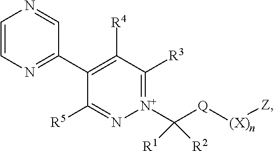 | |

wherein R³, R⁴, R⁵ and Z are as defined in Table 1, R¹ and R² are
hydrogen, Q is (CR^{1a}R^{2b})m, m is 0 and n is 0.

TABLE 11
This table discloses 92 specific compounds of the formula (T-11)

| | |
|---|---|
| | (T-11) |
| 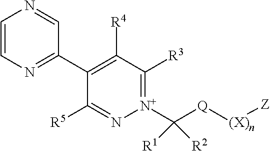 | |

wherein R³, R⁴, R⁵ and Z are as defined in Table 1, R¹ and R² are
hydrogen, Q is CH₂, m is 1 and n is 0.

TABLE 12
This table discloses 92 specific compounds of the formula (T-12)

| | |
|---|---|
| | (T-12) |
| 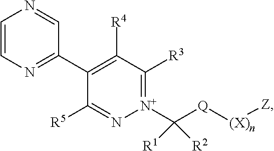 | |

wherein R³, R⁴, R⁵ and Z are as defined in Table 1, R¹ and R² are
hydrogen, Q is CH₂, m is 2 and n is 0.

TABLE 13
This table discloses 92 specific compounds of the formula (T-13)

| | |
|---|---|
| | (T-13) |
| 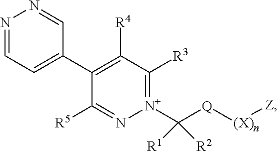 | |

wherein R³, R⁴, R⁵ and Z are as defined in Table 1, R¹ and R² are
hydrogen, Q is (CR^{1a}R^{2b})ₘ, m is 0 and n is 0.

TABLE 14
This table discloses 92 specific compounds of the formula (T-14)

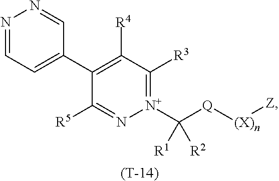

wherein R³, R⁴, R⁵ and Z are as defined in Table 1, R¹ and R² are hydrogen, Q is CH₂, m is 1 and n is 0.

TABLE 15
This table discloses 92 specific compounds of the formula (T-15)

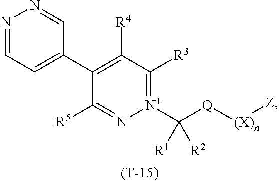

wherein R³, R⁴, R⁵ and Z are as defined in Table 1, R¹ and R² are hydrogen, Q is CH₂, m is 2 and n is 0.

TABLE 16
This table discloses 92 specific compounds of the formula (T-16)

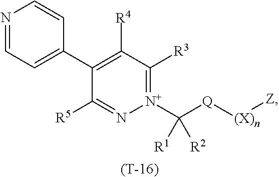

wherein R³, R⁴, R⁵ and Z are as defined in Table 1, R¹ and R² are hydrogen, m is 0 and n is 0.

TABLE 17
This table discloses 92 specific compounds of the formula (T-17)

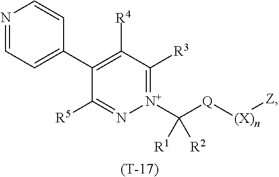

wherein R³, R⁴, R⁵ and Z are as defined in Table 1, R¹ and R² are hydrogen, Q is CH₂, m is 1 and n is 0.

TABLE 18
This table discloses 92 specific compounds of the formula (T-18)

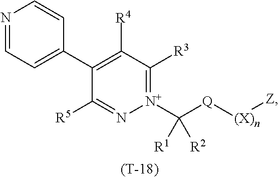

wherein R³, R⁴, R⁵ and Z are as defined in Table 1, R¹ and R² are hydrogen, Q is CH₂, m is 2 and n is 0.

TABLE 19
This table discloses 92 specific compounds of the formula (T-19)

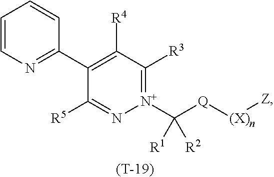

wherein R³, R⁴, R⁵ and Z are as defined in Table 1, R¹ and R² are hydrogen, Q is (CR^{1a}R^{2b})ₘ, m is 0 and n is 0.

TABLE 20
This table discloses 92 specific compounds of the formula (T-20)

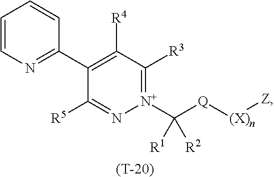

wherein R³, R⁴, R⁵ and Z are as defined in Table 1, R¹ and R² are hydrogen, Q is CH₂, m is 1 and n is 0.

TABLE 21
This table discloses 92 specific compounds of the formula (T-21)

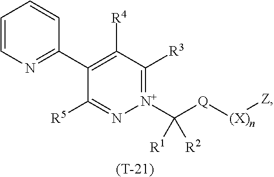

wherein R³, R⁴, R⁵ and Z are as defined in Table 1, R¹ and R² are hydrogen, Q is CH₂, m is 2 and n is 0.

TABLE 22
This table discloses 92 specific compounds of the formula (T-22)

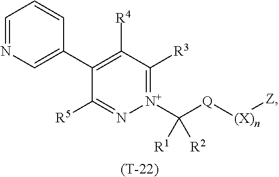

wherein R³, R⁴, R⁵ and Z are as defined in Table 1, R¹ and R² are hydrogen, Q is (CR^{1a}R^{2b})ₘ, m is 0 and n is 0.

TABLE 23
This table discloses 92 specific compounds of the formula (T-23)

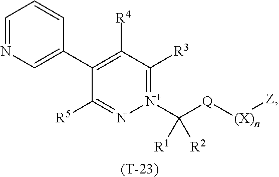

wherein R³, R⁴, R⁵ and Z are as defined in Table 1, R¹ and R² are hydrogen, Q is CH₂, m is 1 and n is 0.

TABLE 24
This table discloses 92 specific compounds of the formula (T-24)

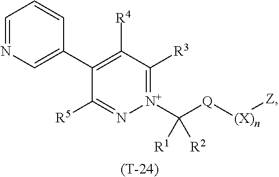

wherein R³, R⁴, R⁵ and Z are as defined in Table 1, R¹ and R² are hydrogen, Q is CH₂, m is 2 and n is 0.

TABLE 25
This table discloses 92 specific compounds of the formula (T-25)

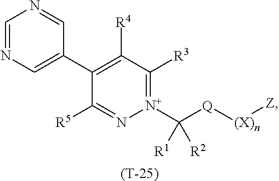

wherein R³, R⁴, R⁵ and Z are as defined in Table 1, R¹ and R² are hydrogen, Q is (CR^{1a}R^{2b})ₘ, m is 0 and n is 0.

TABLE 26
This table discloses 92 specific compounds of the formula (T-26)

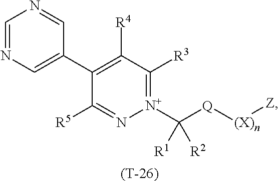

wherein R³, R⁴, R⁵ and Z are as defined in Table 1, R¹ and R² are hydrogen, Q is CH₂, m is 1 and n is 0.

TABLE 27
This table discloses 92 specific compounds of the formula (T-27)

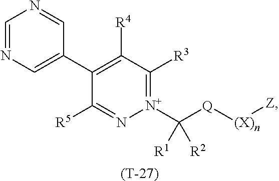

wherein R³, R⁴, R⁵ and Z are as defined in Table 1, R¹ and R² are hydrogen, Q is CH₂, m is 2 and n is 0.

In one set of embodiments, the compound according to Formula (I) is selected from a compound A1 to A7 listed in Table A.

The compounds of the present invention may be prepared according to the following schemes in which the substituents n, m, r, A, Q, X, Z, R¹, R², R^{1a}, R^{2b}, R², R³, R⁴, R⁵, R⁶, R⁷, R^{7a}, R^{7b}, R^{7c}, R⁸, R⁹, R¹⁰, R¹⁵, R^{15a}, R¹⁶, and R¹⁷ are as defined hereinbefore unless explicitly stated otherwise. The compounds of the preceeding Tables 1 to 27 may thus be obtained in an analogous manner.

The compounds of Formula (I) may be prepared by the alkylation of compounds of formula (X), wherein R³, R⁴, R⁵ and A are as defined for compounds of Formula (I), with a suitable alkylating agent of formula (W), wherein R¹, R², Q, X, n and Z are as defined for compounds of Formula (I) and LG is a suitable leaving group, for example, halide or pseudohalide such as triflate, mesylate or tosylate, in a suitable solvent at a suitable temperature, as described in reaction scheme 1. Example conditions include stirring a compound of formula (X) with an alkylating agent of formula (W) in a solvent, or mixture of solvents, such as acetone, dichloromethane, dichloroethane, N,N-dimethylformamide, acetonitrile, 1,4-dioxane, water, acetic acid or trifluroacetic acid at a temperature between −78° C. and 150° C. Alkylating agents of formula (W) are either known in the literature or may be prepared by known literature methods.

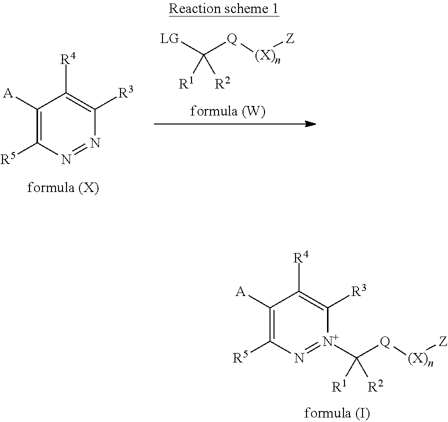

Compounds of Formula (I) may also be prepared by reacting compounds of formula (X), wherein R³, R⁴, R⁵ and A are as defined for compounds of Formula (I), with a suitable alcohol of formula (W), wherein R¹, R², Q, X, n and Z are as defined for compounds of Formula (I), under Mitsunobu-type conditions such as those reported by Petit et al, Tet. Lett. 2008, 49 (22), 3663. Suitable phosphines include triphenylphosphine, suitable azodicarboxylates include diisopropylazodicarboxylate and suitable acids include fluoroboric acid, triflic acid and bis(trifluoromethylsulfonyl)amine, as described in reaction scheme 2. Such alcohols are either known in the literature or may be prepared by known literature methods.

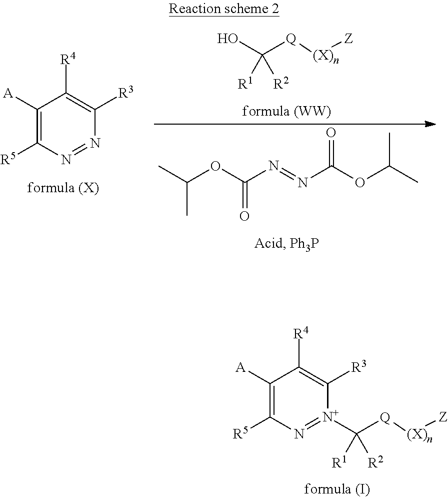

Compounds of Formula (I) may also be prepared by reacting compounds of formula (C), wherein Q, Z, X, n, R¹, R², R³, R⁴, R⁵ and A are as defined for compounds of Formula (I), with a hydrazine of formula (D) in a suitable solvent or mixture of solvents, in the presence of a suitable acid at a suitable temperature, between −78° C. and 150° C., as described in reaction scheme 3. Suitable solvents, or mixtures thereof, include, but are not limited to, alcohols, such as methanol, ethanol and isopropanol, water, aqueous hydrochloric acid, aqueous sulfuric acid, acetic acid and trifluoroacetic acid. Hydrazine compounds of formula (D), for example 2,2-dimethylpropyl 2-hydrazinoethanesulfonate, are either known in the literature or may be prepared by known literature procedures.

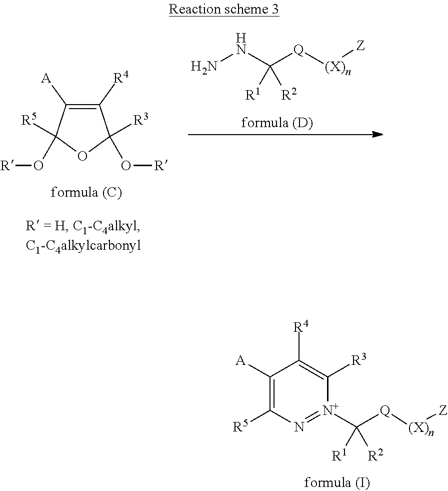

Compounds of formula (C) may be prepared by reacting compounds of formula (G), wherein R³, R⁴, R⁵ and A are as defined for compounds of Formula (I), with an oxidising agent in a suitable solvent at a suitable temperature, between −78° C. and 150° C., optionally in the presence of a suitable base, as described in reaction scheme 4. Suitable oxidising agents include, but are not limited to, bromine and suitable solvents include, but are not limited to alcohols such as methanol, ethanol and isopropanol. Suitable bases include, but are not limited to, sodium bicarbonate, sodium carbonate, potassium bicarbonate, potassium carbonate and potassium acetate. Similar reactions are known in the literature (for example Hufford, D. L.; Tarbell, D. S.; Koszalka, T. R. J. Amer. Chem. Soc., 1952, 3014). Furans of formula (G) are known in the literature or may be prepared using literature methods. Example methods include, but are not limited to, transition metal cross-couplings such as Stille (for example Farina, V.; Krishnamurthy, V.; Scott, W. J. Organic Reactions, Vol. 50. 1997, and Gazzard, L. et al. J. Med. Chem., 2015, 5053), Suzuki-Miyaura (for example Ando, S.; Matsunaga, H.; Ishizuka, T. J. Org. Chem. 2017, 1266-1272, and Ernst, J. B.; Rakers, L.; Glorius, F. Synthesis, 2017, 260), Negishi (for example Yang, Y.; Oldenhius, N. J.; Buchwald, S. L. Angew. Chem. Int. Ed. 2013, 615, and Braendvang, M.; Gundersen, L. Bioorg. Med. Chem. 2005, 6360), and Kumada (for example Heravi, M. M.; Hajiabbasi, P. Monatsh. Chem., 2012, 1575). The coupling partners may be selected with reference to the specific cross-coupling reaction and target product. Transition metal catalysts, ligands, bases, solvents and temperatures may be selected with reference to the desired cross-coupling and are known in the literature. Cross-coupling reactions using pseudo halogens, including but not limited to, triflates, mesylates, tosylates and anisoles, may also be achieved under related conditions.

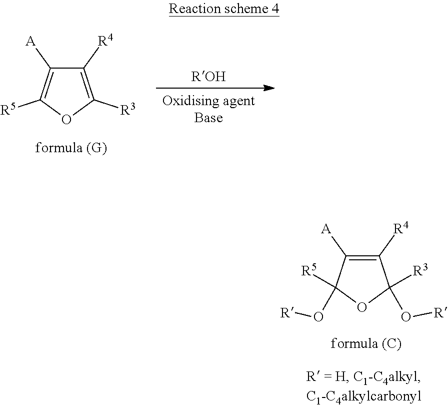

In another approach a compound of Formula (I), wherein Q, Z, X, R¹, R², R³, R⁴, R⁵ and A are as defined for compounds of Formula (I), may be prepared from a compound of formula (R) and an oxidant, in a suitable solvent at a suitable temperature, as outlined in reaction scheme 5. Example oxidants include, but are not limited to, 2,3-dichloro-5,6-dicyano-1,4-benzoquinone, tetrachloro-p-benzoquinone, potassium permanganate, manganese dioxide, 2,2,6,6-tetramethyl-1-piperidinyloxy and bromine. Related reactions are known in the literature.

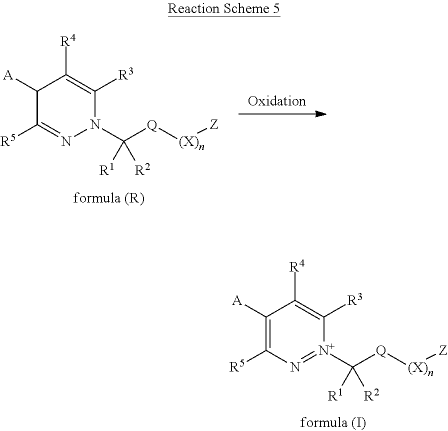

A compound of formula (R), wherein Q, Z, X, R¹, R², R³, R⁴, R⁵ and A are as defined for compounds of Formula (I), may be prepared from a compound of formula (S) and an organometallic of formula (T), which includes, but is not limited to, organomagnesium, organolithium, organocopper and organozinc reagents (M′), in a suitable solvent at a suitable temperature, optionally in the presence of an additional transition metal additive, as outlined in reaction scheme 6. Example conditions include treating a compound of formula (S) with a Grignard of formula (T), in the presence of 0.05-100% copper iodide, in a solvent such as tetrahydrofuran at a temperature between −78° C. and 100° C. Organometallics of formula (T) are known in the literature, or may be prepared by known literature methods. Compounds of formula (S) may be prepared by analogous reactions to those for the preparation of compounds of Formula (I).

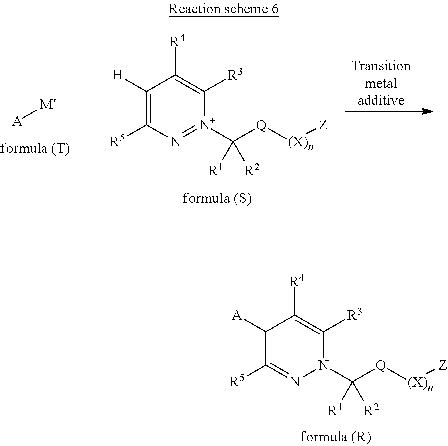

Biaryl pyridazines of formula (X) are known in the literature or may be prepared using literature methods. Example methods include, but are not limited to, the transition metal cross-coupling of compounds of formula (H) and formula (J), or alternatively compounds of formula (K) and formula (L), in which compounds of formula (J) and formula (L) are either an organostannane, organoboronic acid or ester, organotrifluoroborate, organomagnesium, organocopper or organozinc (M′), as outlined in reaction scheme 7. Hal is defined as a halogen or pseudo halogen, for example triflate, mesylate and tosylate. Such cross-couplings include Stille (for example Sauer, J.; Heldmann, D. K. Tetrahedron, 1998, 4297), Suzuki-Miyaura (for example Luebbers, T.; Flohr, A.; Jolidon, S.; David-Pierson, P.; Jacobsen, H.; Ozmen, L.; Baumann, K. Bioorg. Med. Chem. Lett., 2011, 6554), Negishi (for example Imahori, T.; Suzawa, K.; Kondo, Y. Heterocycles, 2008, 1057), and Kumada (for example Heravi, M. M.; Hajiabbasi, P. Monatsh. Chem., 2012, 1575). The coupling partners may be selected with reference to the specific cross-coupling reaction and target product. Transition metal catalysts, ligands, bases, solvents and temperatures may be selected with reference to the desired cross-coupling and are known in the literature. Compounds of formula (H), formula (K) and formula (L) are known in the literature, or may be prepared by known literature methods.

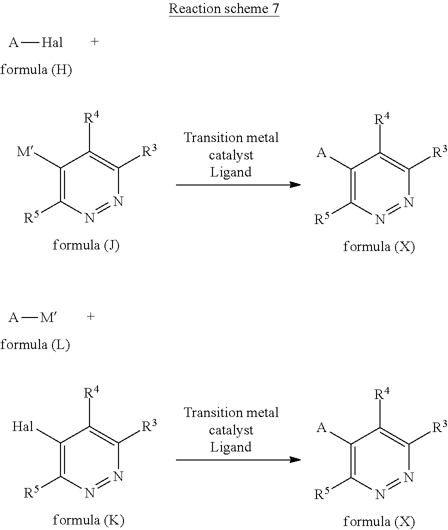

An organometallic of formula (J), which is either an organostannane, organoboronic acid or ester, organotrifluoroborate, organomagnesium, organocopper or organozinc (M′), may be prepared from a compound of formula (XX), wherein R³, R⁴ and R⁵ are as defined for compounds of Formula (I), by metallation, as outlined in reaction scheme 11. Similar reactions are known in the literature (for example Ramphal et al, WO2015153683, Unsinn et al., Organic Letters, 15(5), 1128-1131; 2013, Sadler et al., Organic & Biomolecular Chemistry, 12(37), 7318-7327; 2014. Alternatively, an organometallic of formula (J) may be prepared from compounds of formula (K), wherein R³, R⁴, R⁵ are as defined for compounds of Formula (I), and Hal is defined as a halogen or pseudo halogen, for example triflate, mesylate and tosylate, as described in scheme 8. Example conditions to prepare an organostannane of formula (J) include treatment of a compound of formula (K) with lithium tributyl tin in an appropriate solvent at an appropriate temperature (for example see WO 2010038465). Example conditions to prepare an organoboronic acid or ester of formula (J) include treatment of a compound of formula (K) with bis(pinacolato)diboron, in the presence of an appropriate transition metal catalyst, appropriate ligand, appropriate base, in an appropriate solvent at an appropriate temperature (for example KR 2015135626). Compounds of formula (K) and formula (XX) are either known in the literature or can be prepared by known methods.

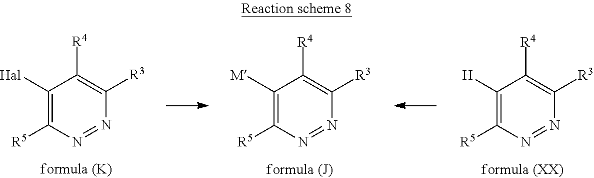

In another approach, an organometallic of formula (J), in which M is either an organostannane or organoboronic acid or ester, may be prepared from a compound of formula (N) and a compound of formula (O), wherein R³, R⁴ and R⁵ are as defined for compounds of Formula (I), as outlined in reaction scheme 9. Examples of such a reaction are known in the literature, for example, Helm et al., Org. and Biomed. Chem., 2006, 4 (23), 4278, Sauer et al., Eur. J. Org. Chem., 1998, 12, 2885, and Helm, M. D.; Moore, J. E.; Plant, A.; Harrity, J. P. A., Angew. Chem. Int. Ed., 2005, 3889. Compounds of formula (N) and formula (O) are known in the literature.

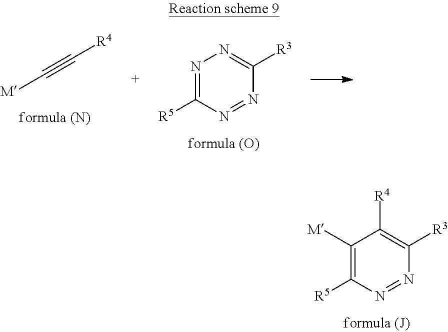

Compounds of formula (X), wherein R³, R⁴, R⁵ and A are as previously defined, may be prepared from compounds of formula (P) and formula (O), in an appropriate solvent, at an appropriate temperature, as outlined in reaction scheme 10. Examples of such a reaction are known in the literature, for example, Sauer et al., Eur. J. Org. Chem., 1998, 12, 2885. Compounds of formula (P) are known in the literature, or may be prepared by known methods.

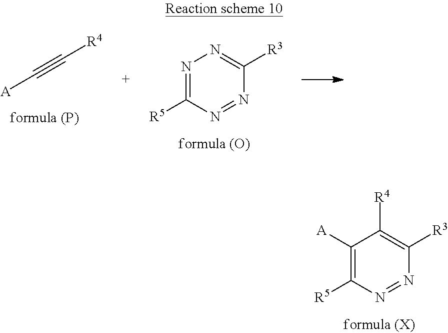

In a further approach a compound of formula (X), wherein R³, R⁴, R⁵ and A are as defined for compounds of Formula (I), may be prepared from compounds of formula (C) and hydrazine, in an appropriate solvent, at an appropriate temperature, as outlined in reaction scheme 11. This reaction may also optionally be performed in the presence of an acid, for example aqueous sulfuric acid or aqueous hydrochloric acid. Similar reactions are known in the literature (for example DE 102005029094, and Chen, B.; Bohnert, T.; Zhou, X.; Dedon, P. C. Chem. Res. Toxicol., 2004, 1406). Compounds of formula (C) may be prepared as previously outlined.

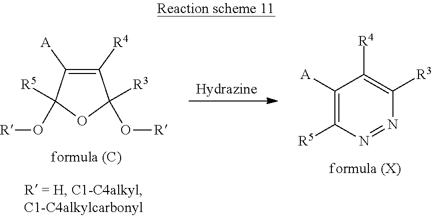

The compounds according to the invention can be used as herbicidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood N.J. (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The herbicidal compositions generally comprise from 0.1 to 99% by weight, especially from 0.1 to 95% by weight, compounds of Formula (I) and from 1 to 99.9% by weight of a formulation adjuvant which preferably includes from 0 to 25% by weight of a surface-active substance. The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 l/ha, especially from 10 to 1000 l/ha.

Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%

The composition of the present may further comprise at least one additional pesticide. For example, the compounds according to the invention can also be used in combination with other herbicides or plant growth regulators. In a preferred embodiment the additional pesticide is a herbicide and/or herbicide safener.

Thus, compounds of Formula (I) can be used in combination with one or more other herbicides to provide various herbicidal mixtures. Specific examples of such mixtures include (wherein “I” represents a compound of Formula (I)):—I+acetochlor; I+acifluorfen (including acifluorfen-sodium); I+aclonifen; I+alachlor; I+alloxydim; I+ametryn; I+amicarbazone; I+amidosulfuron; I+aminocyclopyrachlor; I+aminopyralid; I+amitrole; I+asulam; I+atrazine; I+bensulfuron (including bensulfuron-methyl); I+bentazone; I+bicyclopyrone; I+bilanafos; I+bifenox; I+bispyribac-sodium; I+bixlozone; I+bromacil; I+bromoxynil; I+butachlor; I+butafenacil; I+cafenstrole; I+carfentrazone (including carfentrazone-ethyl); cloransulam (including cloransulam-methyl); I+chlorimuron (including chlorimuron-ethyl); I+chlorotoluron; I+cinosulfuron; I+chlorsulfuron; I+cinmethylin; I+clacyfos; I+clethodim; I+clodinafop (including clodinafop-propargyl); I+clomazone; I+clopyralid; I+cyclopyranil; I+cyclopyrimorate; I+cyclosulfamuron; I+cyhalofop (including cyhalofop-butyl); I+2,4-D (including the choline salt and 2-ethylhexyl ester thereof); I+2,4-DB; I+daimuron; I+desmedipham; I+dicamba (including the aluminum, aminopropyl, bis-aminopropylmethyl, choline, dichloroprop, diglycolamine, dimethylamine, dimethylammonium, potassium and sodium salts thereof); I+diclofop-methyl; I+diclosulam; I+diflufenican; I+difenzoquat; I+diflufenican; I+diflufenzopyr; I+dimethachlor; I+dimethenamid-P; I+diquat dibromide; I+diuron; I+esprocarb; I+ethalfluralin; I+ethofumesate; I+fenoxaprop (including fenoxaprop-P-ethyl); I+fenoxasulfone; I+fenquinotrione; I+fentrazamide; I+flazasulfuron; I+florasulam; I+florpyrauxifen; I+fluazifop (including fluazifop-P-butyl); I+flucarbazone (including flucarbazone-sodium); I+flufenacet; I+flumetralin; I+flumetsulam; I+flumioxazin; I+flupyrsulfuron (including flupyrsulfuron-methyl-sodium); I+fluroxypyr (including fluroxypyr-meptyl); I+fluthiacet-methyl; I+fomesafen; I+foramsulfuron; I+glufosinate (including the ammonium salt thereof); I+glyphosate (including the diammonium, isopropylammonium and potassium salts thereof); I+halauxifen (including halauxifen-methyl); I+halosulfuron-methyl; I+haloxyfop (including haloxyfop-methyl); I+hexazinone; I+hydantocidin; I+imazamox; I+imazapic; I+imazapyr; I+imazaquin; I+imazethapyr; I+indaziflam; I+iodosulfuron (including iodosulfuron-methyl-sodium); I+iofensulfuron: I+iofensulfuron-sodium; I+ioxynil; I+ipfencarbazone; I+isoproturon; I+isoxaben; I+isoxaflutole; I+lactofen; I+lancotrione; I+linuron; I+MCPA; I+MCPB; I+mecoprop-P; I+mefenacet; I+mesosulfuron; I+mesosulfuron-methyl; I+mesotrione; I+metamitron; I+metazachlor; I+methiozolin; I+metobromuron; I+metolachlor; I+metosulam; I+metoxuron; I+metribuzin; I+metsulfuron; I+molinate; I+napropamide; I+nicosulfuron; I+norflurazon; I+orthosulfamuron; I+oxadiargyl; I+oxadiazon; I+oxasulfuron; I+oxyfluorfen; I+paraquat dichloride; I+pendimethalin; I+penoxsulam; I+phenmedipham; I+picloram; I+picolinafen; I+pinoxaden; I+pretilachlor; I+primisulfuron-methyl; I+prodiamine; I+prometryn; I+propachlor; I+propanil; I+propaquizafop; I+propham; I+propyrisulfuron, I+propyzamide; I+prosulfocarb; I+prosulfuron; I+pyraclonil; I+pyraflufen (including pyraflufen-ethyl): I+pyrasulfotole; I+pyrazolynate, I+pyrazosulfuron-ethyl; I+pyribenzoxim; I+pyridate; I+pyriftalid; I+pyrimisulfan, I+pyrithiobac-sodium; I+pyroxasulfone; I+pyroxsulam; I+quinclorac; I+quinmerac; I+quizalofop (including quizalofop-P-ethyl and quizalofop-P-tefuryl); I+rimsulfuron; I+saflufenacil; I+sethoxydim; I+simazine; I+S-metolachlor; I+sulcotrione; I+sulfentrazone; I+sulfosulfuron; I+tebuthiuron; I+tefuryltrione; I+tembotrione; I+terbuthylazine; I+terbutryn; I+thiencarbazone; I+thifensulfuron; I+tiafenacil; I+tolpyralate; I+topramezone; I+tralkoxydim; I+triafamone; I+triallate; I+triasulfuron; I+tribenuron (including tribenuron-methyl); I+triclopyr; I+trifloxysulfuron (including trifloxysulfuron-sodium); I+trifludimoxazin; I+trifluralin; I+triflusulfuron; I+tritosulfuron; I+4-hydroxy-1-methoxy-5-methyl-3-[4-(trifluoromethyl)-2-pyridyl]imidazolidin-2-one; I+4-hydroxy-1,5-dimethyl-3-[4-(trifluoromethyl)-2-pyridyl]imidazolidin-2-one; I+5-ethoxy-4-hydroxy-1-methyl-3-[4-(trifluoromethyl)-2-pyridyl]imidazolidin-2-one; I+4-hydroxy-1-methyl-3-[4-(trifluoromethyl)-2-pyridyl]imidazolidin-2-one; I+4-hydroxy-1,5-dimethyl-3-[1-methyl-5-(trifluoromethyl)pyrazol-3-yl]imidazolidin-2-one; I+(4R)1-(5-tert-butylisoxazol-3-yl)-4-ethoxy-5-hydroxy-3-methyl-imidazolidin-2-one; I+3-[2-(3,4-dimethoxyphenyl)-6-methyl-3-oxo-pyridazine-4-carbonyl]bicyclo[3.2.1]octane-2,4-dione; I+2-[2-(3,4-dimethoxyphenyl)-6-methyl-3-oxo-pyridazine-4-carbonyl]-5-methyl-cyclohexane-1,3-dione; I+2-[2-(3,4-dimethoxyphenyl)-6-methyl-3-oxo-pyridazine-4-carbonyl]cyclohexane-1,3-dione; I+2-[2-(3,4-dimethoxyphenyl)-6-methyl-3-oxo-pyridazine-4-carbonyl]-5,5-dimethyl-cyclohexane-1,3-dione; I+6-[2-(3,4-dimethoxyphenyl)-6-methyl-3-oxo-pyridazine-4-carbonyl]-2,2,4,4-tetramethyl-cyclohexane-1,3,5-trione; I+2-[2-(3,4-dimethoxyphenyl)-6-methyl-3-oxo-pyridazine-4-carbonyl]-5-ethyl-cyclohexane-1,3-dione; I+2-[2-(3,4-dimethoxyphenyl)-6-methyl-3-oxo-pyridazine-4-carbonyl]-4,4,6,6-tetramethyl-cyclohexane-1,3-dione; I+2-[6-cyclopropyl-2-(3,4-dimethoxyphenyl)-3-oxo-pyridazine-4-carbonyl]-5-methyl-cyclohexane-1,3-dione; I+3-[6-cyclopropyl-2-(3,4-dimethoxyphenyl)-3-oxo-pyridazine-4-carbonyl]bicyclo[3.2.1]octane-2,4-dione; I+2-[6-cyclopropyl-2-(3,4-dimethoxyphenyl)-3-oxo-pyridazine-4-carbonyl]-5,5-dimethyl-cyclohexane-1,3-dione; I+6-[6-cyclopropyl-2-(3,4-dimethoxyphenyl)-3-oxo-pyridazine-4-carbonyl]-2,2,4,4-tetramethyl-cyclohexane-1,3,5-trione; I+2-[6-cyclopropyl-2-(3,4-dimethoxyphenyl)-3-oxo-pyridazine-4-carbonyl]cyclohexane-1,3-dione; I+4-[2-(3,4-dimethoxyphenyl)-6-methyl-3-oxo-pyridazine-4-carbonyl]-2,2,6,6-tetramethyl-tetrahydropyran-3,5-dione and I+4-[6-cyclopropyl-2-(3,4-dimethoxyphenyl)-3-oxo-pyridazine-4-carbonyl]-2,2,6,6-tetramethyl-tetrahydropyran-3,5-dione.

The mixing partners of the compound of Formula (I) may also be in the form of esters or salts, as mentioned e.g. in The Pesticide Manual, Fourteenth Edition, British Crop Protection Council, 2006.

The compound of Formula (I) can also be used in mixtures with other agrochemicals such as fungicides, nematicides or insecticides, examples of which are given in The Pesticide Manual.

The mixing ratio of the compound of Formula (I) to the mixing partner is preferably from 1:100 to 1000:1.

The mixtures can advantageously be used in the above-mentioned formulations (in which case “active ingredient” relates to the respective mixture of compound of Formula (I) with the mixing partner).

Compounds of Formula (I) of the present invention may also be combined with herbicide safeners. Preferred combinations (wherein “I” represents a compound of Formula (I)) include:—I+benoxacor, I+cloquintocet (including cloquintocet-mexyl); I+cyprosulfamide; I+dichlormid; I+fenchlorazole (including fenchlorazole-ethyl); I+fenclorim; I+fluxofenim; I+furilazole I+isoxadifen (including isoxadifen-ethyl); I+mefenpyr (including mefenpyr-diethyl); I+metcamifen; I+N-(2-methoxybenzoyl)-4-[(methylaminocarbonyl)amino] benzenesulfonamide and I+oxabetrinil.

Particularly preferred are mixtures of a compound of Formula (I) with cyprosulfamide, isoxadifen (including isoxadifen-ethyl), cloquintocet (including cloquintocet-mexyl) and/or N-(2-methoxybenzoyl)-4-[(methyl-aminocarbonyl)amino]benzenesulfonamide.

The safeners of the compound of Formula (I) may also be in the form of esters or salts, as mentioned e.g. in The Pesticide Manual, 14^{th} Edition (BCPC), 2006. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048, and the reference to fenchlorazole-ethyl also applies to fenchlorazole, etc.

Preferably the mixing ratio of compound of Formula (I) to safener is from 100:1 to 1:10, especially from 20:1 to 1:1.

The mixtures can advantageously be used in the above-mentioned formulations (in which case “active ingredient” relates to the respective mixture of compound of Formula (I) with the safener).

The compounds of Formula (I) of this invention are useful as herbicides. The present invention therefore further comprises a method for controlling unwanted plants comprising applying to the said plants or a locus comprising them, an effective amount of a compound of the invention or a herbicidal composition containing said compound. ‘Controlling’ means killing, reducing or retarding growth or preventing or reducing germination. Generally the plants to be controlled are unwanted plants (weeds). ‘Locus’ means the area in which the plants are growing or will grow.

The rates of application of compounds of Formula (I) may vary within wide limits and depend on the nature of the soil, the method of application (pre-emergence; post-emergence; application to the seed furrow; no tillage application etc.), the crop plant, the weed(s) to be controlled, the prevailing climatic conditions, and other factors governed by the method of application, the time of application and the target crop. The compounds of Formula (I) according to the invention are generally applied at a rate of from 10 to 2000 g/ha, especially from 50 to 1000 g/ha.

The application is generally made by spraying the composition, typically by tractor mounted sprayer for large areas, but other methods such as dusting (for powders), drip or drench can also be used.

Useful plants in which the composition according to the invention can be used include crops such as cereals, for example barley and wheat, cotton, oilseed rape, sunflower, maize, rice, soybeans, sugar beet, sugar cane and turf.

Crop plants can also include trees, such as fruit trees, palm trees, coconut trees or other nuts. Also included are vines such as grapes, fruit bushes, fruit plants and vegetables.

Crops are to be understood as also including those crops which have been rendered tolerant to herbicides or classes of herbicides (e.g. ALS-, GS-, EPSPS-, PPO-, ACCase- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood to include those which are obtained by conventional methods of breeding or genetic engineering and contain so-called output traits (e.g. improved storage stability, higher nutritional value and improved flavour).

Other useful plants include turf grass for example in golf-courses, lawns, parks and roadsides, or grown commercially for sod, and ornamental plants such as flowers or bushes.

Compounds of Formula (I) and compositions of the invention can typically be used to control a wide variety of monocotyledonous and dicotyledonous weed species. Examples of monocotyledonous species that can typically be controlled include Alopecurus myosuroides, Avena fatua, Brachiaria plantaginea, Bromus tectorum, Cyperus esculentus, Digitaria sanguinalis, Echinochloa crus-galli, Lolium perenne, Lolium multiflorum, Panicum miliaceum, Poa annua, Setaria viridis, Setaria faberi and Sorghum bicolor. Examples of dicotyledonous species that can be controlled include Abutilon theophrasti, Amaranthus retroflexus, Bidens pilosa, Chenopodium album, Euphorbia heterophylla, Galium aparine, Ipomoea hederacea, Kochia scoparia, Polygonum convolvulus, Sida spinosa, Sinapis arvensis, Solanum nigrum, Stellaria media, Veronica persica and Xanthium strumarium.

Compounds/compositions of the invention are particularly useful in non-selective burn-down applications, and as such may also be used to control volunteer or escape crop plants.

Various aspects and embodiments of the present invention will now be illustrated in more detail by way of example. It will be appreciated that modification of detail may be made without departing from the scope of the invention.

### EXAMPLES

The Examples which follow serve to illustrate, but do not limit, the invention.

### FORMULATION EXAMPLES

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

Emulsifiable concentrate

| | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill.

Extruder granules

| | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

Coated granules

| | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

Suspension concentrate

| | |
|---|---|
| active ingredients | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### List of Abbreviations

Boc=tert-butyloxycarbonyl
br=broad
CDCl₃=chloroform-d
CD₃OD=methanol-d
° C.=degrees Celsius
D₂O=water-d
DCM=dichloromethane
d=doublet
dd=double doublet
dt=double triplet
DMSO=dimethylsulfoxide
EtOAc=ethyl acetate
h=hour(s)
HCl=hydrochloric acid
HPLC=high-performance liquid chromatography (description of the apparatus and the methods used for HPLC are given below)
m=multiplet
M=molar
min=minutes
MHz=mega hertz
mL=millilitre
mp=melting point
ppm=parts per million
q=quartet
quin=quintet
rt=room temperature
s=singlet
t=triplet
THF=tetrahydrofuran

### LC/MS=Liquid Chromatography Mass Spectrometry

### Preparative Reverse Phase HPLC Method

Compounds purified by mass directed preparative HPLC using ES+/ES− on a Waters FractionLynx Autopurification system comprising a 2767 injector/collector with a 2545 gradient pump, two 515 isocratic pumps, SFO, 2998 photodiode array (Wavelength range (nm): 210 to 400), 2424 ELSD and QDa mass spectrometer. A Waters Atlantis T3 5 micron 19×10 mm guard column was used with a Waters Atlantis T3 OBD, 5 micron 30×100 mm prep column.

### Ionisation Method

Electrospray positive and negative: Cone (V) 20.00, Source Temperature (° C.) 120, Cone Gas Flow (L/Hr.) 50

Mass range (Da): positive 100 to 800, negative 115 to 800.

The preparative HPLC was conducted using an 11.4 minute run time (not using at column dilution, bypassed with the column selector), according to the following gradient table:

| Time (mins) | Solvent A (%) | Solvent B (%) | Flow (ml/min) |
|---|---|---|---|
| 0.00 | 100 | 0 | 35 |
| 2.00 | 100 | 0 | 35 |
| 2.01 | 100 | 0 | 35 |
| 7.0 | 90 | 10 | 35 |
| 7.3 | 0 | 100 | 35 |
| 9.2 | 0 | 100 | 35 |
| 9.8 | 99 | 1 | 35 |
| 11.35 | 99 | 1 | 35 |
| 11.40 | 99 | 1 | 35 |

515 pump 0 ml/min Acetonitrile (ACD)
515 pump 1 ml/min 90% Methanol/10% Water (make up pump)
Solvent A: Water with 0.05% Trifluoroacetic Acid
Solvent B: Acetonitrile with 0.05% Trifluoroacetic Acid

### PREPARATION EXAMPLES

### Example 1: Preparation of 5-[2-(4-pyrimidin-2-ylpyridazin-1-ium-1-yl)ethyl]thiazolidine-2,4-dione trifluoroacetate A1

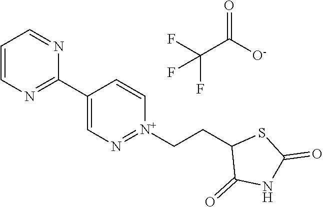 [SMILES: O=C1NC(=O)C(CC[n+]2ccc(-c3ncccn3)cn2)S1 ; O=C([O-])C(F)(F)F]

### Step 1: Preparation of tributyl(pyridazin-4-yl)stannane

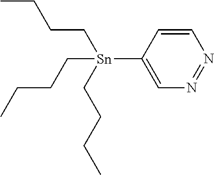 [SMILES: CCC[CH2][Sn]([CH2]CCC)([CH2]CCC)[c]1ccnnc1]

To a solution of lithium diisopropylamide (1M solution in tetrahydrofuran, 125 mL) at −78° C., under nitrogen atmosphere, was added a solution of pyridazine (10 g) and tri-n-butyltin chloride (44.6 g) in tetrahydrofuran (100 mL) drop wise. The reaction mixture was stirred at −78° C. for 1 hour. The reaction mixture was warmed to room temperature and quenched with saturated aqueous ammonium chloride (100 mL) and extracted with ethyl acetate (3×150 mL). The organic layer was dried over sodium sulfate, concentrated and purified by chromatography on silica eluting with 30% ethyl acetate in hexanes to afford tributyl(pyridazin-4-yl)stannane as a pale brown liquid.

¹H NMR (400 MHz, CDCl₃) 9.17 (t, 1H) 9.02 (dd, 1H) 7.54 (dd, 1H) 1.57-1.49 (m, 6H) 1.37-1.29 (m, 6H) 1.19-1.13 (m, 6H) 0.92-0.86 (m, 9H).

### Step 2: Preparation of 2-pyridazin-4-ylpyrimidine

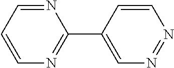 [SMILES: c1cnc(-c2ccnnc2)nc1]

A solution of 2-bromopyrimidine (2.5 g) and tributyl(pyridazin-4-yl)stannane (5.8 g) in tetrahydrofuran (25 mL) was degassed with argon for 20 minutes. Tetrakis (triphenylphosphine) palladium (0) (1.8 g) was added at room temperature and the mixture was heated under microwave irradiation at 120° C. for 30 minutes. The reaction mixture was poured into water and extracted with ethyl acetate (100 mL). The organic layer was concentrated and purified by chromatography on silica eluting with 80% ethyl acetate in hexanes to give 2-pyridazin-4-ylpyrimidine as a beige solid.

¹H NMR (400 MHz, CDCl₃) 10.17 (dd, 1H) 9.39 (dd, 1H) 8.92 (d, 2H) 8.43 (dd, 1H) 7.39 (t, 1H)

### Step 3: Preparation of 5-(2-hydroxyethyl)thiazolidine-2,4-dione

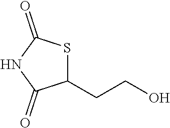

To a solution of thiazolidine-2,4-dione (1 g) in anhydrous tetrahydrofuran (50 mL), under a nitrogen atmosphere at −78° C., was added n-butyllithium (7.2 mL) drop wise. The reaction mixture was allowed to warm to 0° C. then was stirred at this temperature for a further 1.5 hours. After cooling again to −78° C. a solution of ethylene oxide (2.8 mL) was added drop wise, followed by warming to room temperature and stirring for a further 4 hours. The reaction mixture was quenched with 1M aqueous hydrochloric acid and extracted with diethyl ether (×3). The combined organic phases were dried over magnesium sulfate then concentrated. The crude product was purified by silica gel chromatography eluting with 0 to 100% ethyl acetate in cyclohexane to afford 5-(2-hydroxyethyl)thiazolidine-2,4-dione as a solid.

¹H NMR (400 MHz, CD₃OD) 4.48 (dd, 1H), 3.82-3.71 (m, 1H), 3.70-3.60 (m, 1H), 2.45-2.31 (m, 1H), 2.12-2.00 (m, 1H)

### Step 4: Preparation of 5-(2-bromoethyl)thiazolidine-2,4-dione

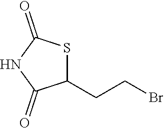

To a solution of 5-(2-hydroxyethyl)thiazolidine-2,4-dione (0.333 g) in tetrahydrofuran (12 mL) was added triphenylphosphine (0.813 g) and carbon tetrabromide (1.03 g). After stirring at room temperature for 3 hours the resulting precipitate was removed by filtration and the filtrate was concentrated. The crude product was purified by silica gel chromatography eluting with 0 to 50% ethyl acetate to afford 5-(2-bromoethyl)thiazolidine-2,4-dione as a colourless gum.

¹H NMR (400 MHz, CDCl₃) 4.55 (dd, 1H), 3.71-3.58 (m, 1H), 3.53-3.42 (m, 2H), 2.83-2.70 (m, 1H), 2.49-2.33 (m, 1H)

### Step 5: Preparation of 5-[2-(4-pyrimidin-2-ylpyridazin-1-ium-1-yl)ethyl]thiazolidine-2,4-dione trifluoroacetate A1

To a mixture of 2-pyridazin-4-ylpyrimidine (0.1 g) and 5-(2-bromoethyl)thiazolidine-2,4-dione (0.283 g) in acetonitrile (6.32 mL) was added sodium iodide (0.095 g) and the reaction mixture was heated at 80° C. overnight. After cooling to room temperature, the reaction was concentrated and the residue was partitioned between water (10 mL) and dichloromethane (20 mL). The aqueous phase was filtered and purified by preparative reverse phase HPLC (trifluoroacetic acid is present in the eluent) to afford 5-[2-(4-pyrimidin-2-ylpyridazin-1-ium-1-yl)ethyl]thiazolidine-2,4-dione trifluoroacetate as a yellow gum.

¹H NMR (400 MHz, CD₃OD) 10.42-10.37 (m, 1H), 10.00 (d, 1H), 9.43 (dd, 1H), 9.13 (d, 2H), 7.71 (t, 1H), 5.17 (t, 2H), 4.63 (t, 1H), 3.02-2.89 (m, 1H), 2.89-2.77 (m, 1H) (NH proton missing)

### Example 2: Preparation of 4-hydroxy-6-methyl-3-[2-(4-pyrimidin-2-ylpyridazin-1-ium-1-yl)acetyl]pyran-2-one trifluoroacetate A2

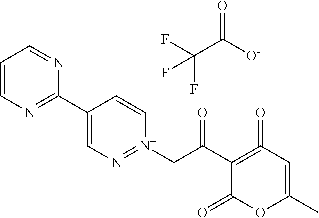

To a solution of 2-pyridazin-4-ylpyrimidine (0.2 g) in acetonitrile (6 mL) was added 3-(2-bromoacetyl)-4-hydroxy-6-methyl-2H-pyran-2-one (0.375 g) and the mixture was heated at 80° C. overnight. The reaction mixture was concentrated and purified by reverse phase preparative HPLC (trifluoroacetic acid is present in the eluent) to afford 4-hydroxy-6-methyl-3-[2-(4-pyrimidin-2-ylpyridazin-1-ium-1-yl)acetyl]pyran-2-one trifluoroacetate as a burgundy solid.

¹H NMR (400 MHz, D₂O) 10.16-10.24 (m, 1H), 9.71 (dd, 1H), 9.28 (dd, 1H), 8.96-9.02 (m, 2H), 7.64 (t, 1H), 6.11-6.24 (m, 1H), 2.24 (s, 3H) (OH and 2 x CH₂C(O) protons missing)

### Example 3: Preparation of 2-bromo-N-(1-methyltetrazol-5-yl)acetamide

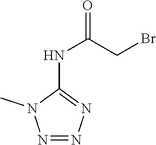

To a solution of 5-amino-1-methyl-1 h-tetrazole (1 g) in toluene (59.9 mL) was added bromoacetyl bromide (3.54 mL) drop wise at room temperature. The reaction was then heated at reflux for 5 hours. The reaction was cooled and the resulting precipitate was filtered off, triturated with dichloromethane and dried to give 2-bromo-N-(1-methyltetrazol-5-yl)acetamide as a pale brown solid, which was used without further purification.

¹H NMR (400 MHz, CD₃OD) 4.11 (s, 2H), 3.97 (s, 3H) (NH proton missing)

### Example 4: Preparation of 5-[(4-pyrimidin-2-ylpyridazin-1-ium-1-yl)methyl]isoxazol-3-ol 2,2,2-trifluoroacetate A9

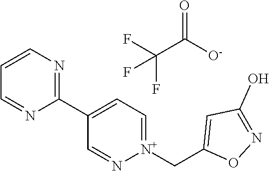 [SMILES: Oc1cc(C[n+]2ccc(-c3ncccn3)cn2)on1 ; O=C([O-])C(F)(F)F]

### Step 1: Preparation of 3-benzyloxy-5-[(4-pyrimidin-2-ylpyridazin-1-ium-1-yl)methyl]isoxazole 2,2,2-trifluoroacetate

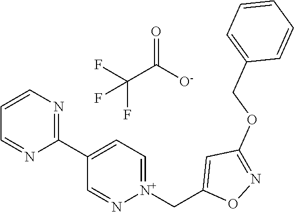 [SMILES: c1ccc(COc2cc(C[n+]3ccc(-c4ncccn4)cn3)on2)cc1 ; O=C([O-])C(F)(F)F]

To a solution of (3-benzyloxyisoxazol-5-yl)methanol (1 g) and 2,6-dimethylpyridine (0.679 mL) in dichloromethane (24.4 mL), cooled to ˜0° C. under nitrogen atmosphere, was added trifluoromethylsulfonyl trifluoromethanesulfonate (0.902 mL) and the mixture was stirred at ˜0° C. for 2 hours. To this was added 2-pyridazin-4-ylpyrimidine (0.771 g) and the mixture was allowed to warm to room temperature and stirred overnight. The reaction mixture was concentrated and purified by preparative reverse phase HPLC (trifluoroacetic acid is present in the eluent) to give 3-benzyloxy-5-[(4-pyrimidin-2-ylpyridazin-1-ium-1-yl)methyl]isoxazole 2,2,2-trifluoroacetate as a yellow solid.

¹H NMR (400 MHz, CD₃OD) 10.39 (d, 1H), 10.08 (dd, 1H), 9.46 (dd, 1H), 9.11 (d, 2H), 7.70 (t, 1H), 7.46-7.29 (m, 5H), 6.53 (s, 1H), 6.27 (s, 2H), 5.26 (s, 2H)

### Step 2: Preparation of 5-[(4-pyrimidin-2-ylpyridazin-1-ium-1-yl)methyl]isoxazol-3-ol 2,2,2-trifluoroacetate A9

To a solution of 3-benzyloxy-5-[(4-pyrimidin-2-ylpyridazin-1-ium-1-yl)methyl]isoxazole 2,2,2-trifluoroacetate (0.2 g) in acetonitrile (2.35 mL), under nitrogen atmosphere, was added iodo(trimethyl)silane (0.62 mL). The reaction mixture was stirred at room temperature for 6 hours. The reaction mixture was quenched with water, concentrated and purified by preparative reverse phase HPLC (trifluoroacetic acid is present in the eluent) to give 5-[(4-pyrimidin-2-ylpyridazin-1-ium-1-yl)methyl]isoxazol-3-ol 2,2,2-trifluoroacetate as an orange gum.

¹H NMR (400 MHz, CD₃OD) 10.40 (d, 1H), 10.08 (d, 1H), 9.47 (dd, 1H), 9.12 (d, 2H), 7.71 (t, 1H), 6.39 (s, 1H), 6.24 (s, 2H) (OH proton missing)

### Example 5: Preparation of 4-pyrimidin-2-yl-1-[2-(1H-triazol-5-ylsulfonyl)ethyl]pyridazin-1-ium 2,2,2-trifluoroacetate A8

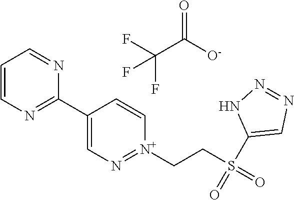 [SMILES: O=S(=O)(CC[n+]1ccc(-c2ncccn2)cn1)c1cnn[nH]1 ; O=C([O-])C(F)(F)F]

To a cooled (˜0° C.) mixture of 2-pyridazin-4-ylpyrimidine (0.1 g) in dry acetonitrile (6.32 mL) was added 1,1,1-trifluoro-N-(trifluoromethylsulfonyl)methanesulfonamide (0.131 mL). The mixture was warmed to room temperature and stirred for 15 minutes. To this was added triphenylphosphine (0.332 g) and 2-(1H-triazol-5-ylsulfonyl)ethanol (0.134 g) followed by drop wise addition of diisopropyl azodicarboxylate (0.249 mL). The reaction mixture was heated at 80° C. for 72 hours. The mixture was cooled, concentrated and the residue partitioned between water and dichloromethane. The aqueous phase was purified by preparative reverse phase HPLC (trifluoroacetic acid is present in the eluent) to give 4-pyrimidin-2-yl-1-[2-(1H-triazol-5-ylsulfonyl)ethyl]pyridazin-1-ium 2,2,2-trifluoroacetate as a brown solid.

¹H NMR (400 MHz, D₂O) 10.14 (d, 1H), 9.96-9.92 (m, 1H), 9.26-9.21 (m, 1H), 9.01 (d, 2H), 8.57-8.55 (m, 1H), 7.65 (t, 1H), 5.39-5.33 (m, 2H), 4.42-4.37 (m, 2H) (NH proton missing)

Additional compounds in Table A (below) were prepared by analogous procedures, from appropriate starting materials. The skilled person would understand that the compounds of Formula (I) may exist as an agronomically acceptable salt, a zwitterion or an agronomically acceptable salt of a zwitterion as described hereinbefore. Where mentioned the specific counterion is not considered to be limiting, and the compound of Formula (e) may be formed with any suitable counter ion.

NMR spectra contained herein were recorded on either a 400 MHz Bruker AVANCE III HO equipped with a Bruker SMART probe unless otherwise stated. Chemical shifts are expressed as ppm downfield from TMS, with an internal reference of either TMS or the residual solvent signals. The following multiplicities are used to describe the peaks: s=singlet, d=doublet, t=triplet, dd=double doublet, dt=double triplet, q=quartet, quin=quintet, m=multiplet. Additionally br. is used to describe a broad signal and app. is used to describe and apparent multiplicity.

TABLE A
Physical Data for Compounds of the Invention

| Compound | | |
|---|---|---|
| Number | Structure | ¹H NMR |
| A1 | 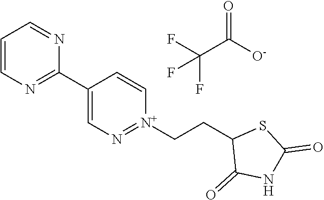 [SMILES: O=C1NC(=O)C(CC[n+]2ccc(-c3ncccn3)cn2)S1 ; O=C([O-])C(F)(F)F] | (400 MHz, CD₃OD) 10.42-10.37 (m, 1H), 10.00 (d, 1H), 9.43 (dd, 1H), 9.13 (d, 2H), 7.71 (t, 1H), 5.17 (t, 2H), 4.63 (t, 1H), 3.02- 2.89 (m, 1H), 2.89-2.77 (m, 1H) (NH proton missing) |
| A2 | 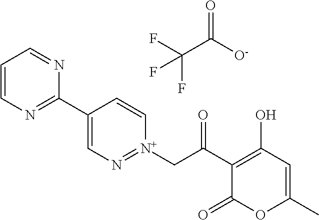 [SMILES: Cc1cc(O)c(C(=O)C[n+]2ccc(-c3ncccn3)cn2)c(=O)o1 ; O=C([O-])C(F)(F)F] | (400 MHz, D₂O) 10.16-10.24 (m, 1H), 9.71 (dd, 1H), 9.28 (dd, 1H), 8.96-9.02 (m, 2H), 7.64 (t, 1H), 6.11-6.24 (m, 1H), 2.24 (s, 3H) (OH and 2 x CH₂C(O) protons missing) |
| A3 | 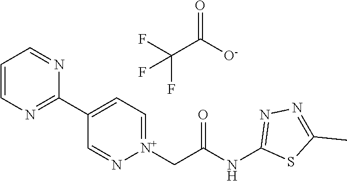 [SMILES: Cc1nnc(NC(=O)C[n+]2ccc(-c3ncccn3)cn2)s1 ; O=C([O-])C(F)(F)F] | (400 MHz, D₂O) 10.27-10.23 (m, 1 H), 9.89 (d, 1H), 9.37-9.32 (m, 1H), 9.10 (dd, 2H), 7.69-7.63 (m, 1H), 6.07-6.02 (m, 1H) 2.56 (s, 3H) (NH and CH protons missing) |
| A4 | 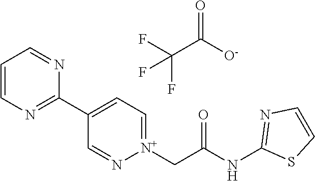 [SMILES: O=C(C[n+]1ccc(-c2ncccn2)cn1)Nc1nccs1 ; O=C([O-])C(F)(F)F] | (400 MHz, D₂O) 10.24-10.14 (m, 1H), 9.87- 9.81 (m, 1H), 9.28 (dd, 1H), 8.96 (d, 2H), 7.66-7.56 (m, 1H), 7.38-7.33 (m, 1H), 7.12 (d, 1H), 5.97 (d, 1H) (NH and CH protons missing) |
| A5 | 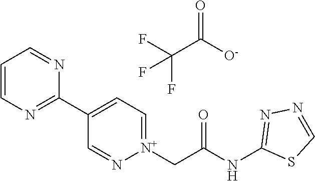 [SMILES: O=C(C[n+]1ccc(-c2ncccn2)cn1)Nc1nncs1 ; O=C([O-])C(F)(F)F] | (400 MHz, D₂O) 10.27-10.19 (m, 1H), 9.89 (dd, 1H), 9.28-9.36 (m, 1H), 9.05-8.95 (m, 3H), 7.66-7.62 (m, 1H), 6.11-6.01 (m, 2H) (NH proton missing) |
| A6 | 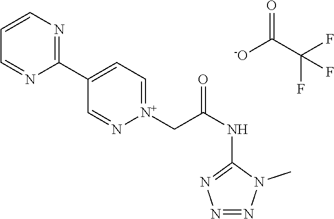 | (400 MHz, D₂O) 10.26-10.21 (m, 1H), 9.88 (dd, 1H), 9.32 (dd, 1H), 9.01 (d, 2H), 7.66 (t, 1H), 6.11 (s, 2H), 3.95 (s, 3H) (NH proton missing) |
| A7 | 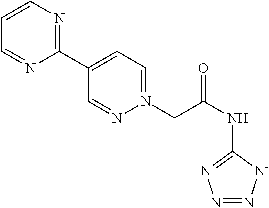 [SMILES: O=C(C[n+]1ccc(-c2ncccn2)cn1)Nc1nnn[n-]1] | (400 MHz, D₂O) 10.24 (s, 1H), 9.89 (d, 1H), 9.33 (dd, 1H), 9.02 (d, 2H), 7.66 (t, 1H), 6.06 (s, 2H) (NH proton missing) |
| A8 | 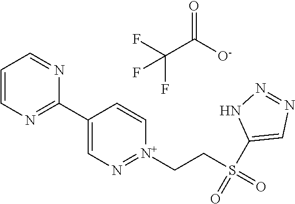 [SMILES: O=S(=O)(CC[n+]1ccc(-c2ncccn2)cn1)c1cnn[nH]1 ; O=C([O-])C(F)(F)F] | (400 MHz, D₂O) 10.14 (d, 1H), 9.96-9.92 (m, 1H), 9.26-9.21 (m, 1H), 9.01 (d, 2H), 8.57- 8.55 (m, 1H), 7.65 (t, 1H), 5.39-5.33 (m, 2H), 4.42-4.37 (m, 2H) (NH proton missing) |
| A9 | 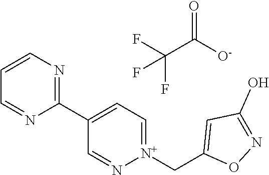 [SMILES: Oc1cc(C[n+]2ccc(-c3ncccn3)cn2)on1 ; O=C([O-])C(F)(F)F] | (400 MHz, CD₃OD) 10.40 (d, 1H), 10.08 (d, 1H), 9.47 (dd, 1H), 9.12 (d, 2H), 7.71 (t, 1H), 6.39 (s, 1H), 6.24 (s, 2H) (OH proton missing) |

### BIOLOGICAL EXAMPLES

### Post-Emergence Efficacy

Seeds of a variety of test species were sown in standard soil in pots. After cultivation for 14 days (post-emergence) under controlled conditions in a glasshouse (at 24/16° C., day/night; 14 hours light; 65% humidity), the plants were sprayed with an aqueous spray solution derived from the dissolution of the technical active ingredient formula (I) in a small amount of acetone and a special solvent and emulsifier mixture referred to as IF50 (11.12% Emulsogen EL360 TM+44.44% N-methylpyrrolidone+44.44% Dowanol DPM glycol ether), to create a 50 g/l solution which was then diluted to required concentration using 0.25% or 1% Empicol ESC70 (Sodium lauryl ether sulphate)+1% ammonium sulphate as diluent.

The test plants were then grown in a glasshouse under controlled conditions (at 24/16° C., day/night; 14 hours light; 65% humidity) and watered twice daily. After 13 days the test was evaluated (100=total damage to plant; 0=no damage to plant).

### Test Plants:

Ipomoea hederacea (IPOHE), Euphorbia heterophylla (EPHHL), Chenopodium album (CHEAL), Amaranthus palmeri (AMAPA), Lolium perenne (LOLPE), Digitaria sanguinalis (DIGSA), Eleusine indica (ELEIN), Echinochloa crus-galli (ECHCG), Setaria faberi (SETFA)

TABLE B
Control of weed species by compounds of Formula
(I) after post-emergence application

| Compound | Application | | | | | | | | | |
|---|---|---|---|---|---|---|---|---|---|---|
| Number | Rate g/Ha | AMAPA | CHEAL | EPHHL | IPOHE | ELEIN | LOLPE | DIGSA | SETFA | ECHCG |
| A1 | 500 | 90 | 30 | 50 | 50 | 70 | 40 | 60 | 60 | 50 |
| A2 | 500 | 90 | 0 | 70 | 10 | 70 | 10 | 30 | 20 | 20 |
| A3 | 500 | 50 | 30 | — | — | 0 | 0 | 0 | 0 | 10 |
| A4 | 500 | 70 | 60 | 100 | 10 | 80 | 0 | 10 | 20 | 0 |
| A5 | 500 | 100 | 30 | 20 | 20 | 70 | 0 | 70 | 20 | 50 |
| A6 | 500 | 90 | 90 | 80 | 40 | 20 | 0 | 30 | 30 | 30 |
| A7 | 500 | 90 | 90 | 30 | 30 | 30 | 10 | 30 | 50 | 20 |
| A8 | 130 | 60 | 0 | 70 | 20 | 50 | 0 | — | 50 | 40 |
| A9 | 500 | 20 | 40 | 30 | 20 | 20 | 10 | 40 | 20 | 30 |

1. A compound of Formula (I) or an agronomically acceptable salt or zwitterionic species thereof: 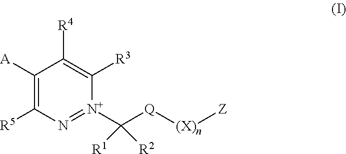
wherein
R¹ is selected from the group consisting of hydrogen, halogen, C₁-C₆alkyl, C₂-C₆alkenyl, C₂-C₆alkynyl, C₃-C₆cycloalkyl, C₁-C₆haloalkyl, —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵;
R² is selected from the group consisting of hydrogen, halogen, C₁-C₆alkyl and C₁-C₆haloalkyl;
and wherein when R¹ is selected from the group consisting of —OR⁷, —OR^{15a}, —N(R⁶)S(O)₂R¹⁵, —N(R⁶)C(O)R¹⁵, —N(R⁶)C(O)OR¹⁵, —N(R⁶)C(O)NR¹⁶R¹⁷, —N(R⁶)CHO, —N(R^{7a})₂ and —S(O)ᵣR¹⁵, R² is selected from the group consisting of hydrogen and C₁-C₆alkyl; or
R¹ and R² together with the carbon atom to which they are attached form a C₃-C₆cycloalkyl ring or a 3- to 6-membered heterocyclyl, which comprises 1 or 2 heteroatoms individually selected from N and O;
Q is (CR^{1a}R^{2b})ₘ;
m is 0, 1, 2 or 3;
each R^{1a} and R^{2b} are independently selected from the group consisting of hydrogen, halogen, C₁-C₆alkyl, C₁-C₆haloalkyl, —OH, —OR⁷, —OR^{15a}, —NH₂, —NHR⁷, —NHR^{15a}, —N(R⁶)CHO, —NR^{7b}R^{7c} and —S(O)ᵣR¹⁵; or
each R^{1a} and R^{2b} together with the carbon atom to which they are attached form a C₃-C₆cycloalkyl ring or a 3- to 6-membered heterocyclyl, which comprises 1 or 2 heteroatoms individually selected from N and O; and
R³, R⁴ and R⁵ are independently selected from the group consisting of hydrogen, halogen, cyano, nitro, —S(O)ᵣR¹⁵, C₁-C₆alkyl, C₁-C₆fluoroalkyl, C₁-C₆fluoroalkoxy, C₁-C₆alkoxy, C₃-C₆cycloalkyl and —N(R⁶)₂;
each R⁶ is independently selected from hydrogen and C₁-C₆alkyl;
each R⁷ is independently selected from the group consisting of C₁-C₆alkyl, —S(O)₂R¹⁵, —C(O)R¹⁵, —C(O)OR¹⁵ and —C(O)NR¹⁶R¹⁷;
each R^{7a} is independently selected from the group consisting of —S(O)₂R¹⁵, —C(O)R¹⁵, —C(O)OR¹⁵, —C(O)NR¹⁶R¹⁷ and —C(O)NR⁶R^{15a};
R^{7b} and R^{7c} are independently selected from the group consisting of C₁-C₆alkyl, —S(O)₂R¹⁵, —C(O)R¹⁵, —C(O)OR¹⁵, —C(O)NR¹⁶R¹⁷ and phenyl, and wherein said phenyl is optionally substituted by 1, 2 or 3 R⁹ substituents, which may be the same or different; or
R^{7b} and R^{7c} together with the nitrogen atom to which they are attached form a 4- to 6-membered heterocyclyl ring which optionally comprises one additional heteroatom individually selected from N, O and S; and
A is a 6-membered heteroaryl, which comprises 1, 2, 3 or 4 nitrogen atoms and wherein the heteroaryl may be optionally substituted by 1, 2, 3 or 4 R⁸ substituents, which may be the same or different,
and wherein when A is substituted by 1 or 2 substituents, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl, C₁-C₆haloalkyl, C₃-C₆cycloalkyl, C₃-C₆halocycloalkyl, C₃-C₆cycloalkoxy, C₂-C₆alkenyl, C₂-C₆haloalkenyl, C₂-C₆alkynyl, C₁-C₃alkoxyC₁-C₃alkyl-, hydroxyC₁-C₆alkyl-, C₁-C₃alkoxyC₁-C₃alkoxy-, C₁-C₆haloalkoxy, C₁-C₃haloalkoxyC₁-C₃alkyl-, C₃-C₆alkenyloxy, C₃-C₆alkynyloxy, N—C₃-C₆cycloalkylamino, —C(R⁶)═NOR⁶, phenyl, a 3- to 6-membered heterocyclyl, which comprises 1 or 2 heteroatoms individually selected from N and O, and a 5- or 6-membered heteroaryl, which comprises 1, 2, 3 or 4 heteroatoms individually selected from N, O and S, and wherein said phenyl, heterocyclyl or heteroaryl are optionally substituted by 1, 2 or 3 R⁹ substituents, which may be the same or different;
and wherein when A is substituted by 3 or 4 substituents, each R⁸ is independently selected from the group consisting of halogen, —NH₂, —NHR⁷, —N(R⁷)₂, —OH, —OR⁷, —C(O)NR¹⁶R¹⁷, —S(O)₂NR¹⁶R¹⁷, C₁-C₆alkyl and C₁-C₆haloalkyl; and
each R⁹ is independently selected from the group consisting of halogen, cyano, nitro, C₁-C₄alkyl, C₁-C₄alkoxy, C₁-C₄haloalkyl and C₁-C₄haloalkoxy;
X is selected from the group consisting of C₃-C₆cycloalkyl, phenyl, a 5- or 6-membered heteroaryl, which comprises 1, 2, 3 or 4 heteroatoms individually selected from N, O and S, and a 4- to 6-membered heterocyclyl, which comprises 1, 2 or 3 heteroatoms individually selected from N, O and S, and wherein said cycloalkyl, phenyl, heteroaryl or heterocyclyl moieties are optionally substituted by 1 or 2 R⁹ substituents, and wherein the aforementioned CR¹R², Q and Z moieties may be attached at any position of said cycloalkyl, phenyl, heteroaryl or heterocyclyl moieties;
n is 0 or 1;
Z is an organic acid comprising: a terminal cyclic moiety other than phenyl, at least one oxygen atom, and at least one further heteroatom independently selected from O, N and S;
R¹⁰ is selected from the group consisting of hydrogen, C₁-C₆alkyl, phenyl and benzyl, and wherein said phenyl or benzyl are optionally substituted by 1, 2 or 3 R⁹ substituents, which may be the same or different;
R¹⁵ is selected from the group consisting of C₁-C₆alkyl and phenyl, and wherein said phenyl is optionally substituted by 1, 2 or 3 R⁹ substituents, which may be the same or different;
R^{15a} is phenyl, wherein said phenyl is optionally substituted by 1, 2 or 3 R⁹ substituents, which may be the same or different;
R¹⁶ and R¹⁷ are independently selected from the group consisting of hydrogen and C₁-C₆alkyl; or
R¹⁶ and R¹⁷ together with the nitrogen atom to which they are attached form a 4- to 6-membered heterocyclyl ring which optionally comprises one additional heteroatom individually selected from N, O and S; and
r is 0, 1 or 2.

2. The compound according to claim 1, wherein R¹ and R² are independently selected from the group consisting of hydrogen and C₁-C₆alkyl.

3. The compound according to claim 1, wherein each R^{1a} and R^{2b} are independently selected from the group consisting of hydrogen, C₁-C₆alkyl, —OH and —NH₂.

4. The compound according to claim 1, wherein m is 1 or 2.

5. The compound according to claim 1, wherein R³, R⁴ and R⁵ are independently selected from the group consisting of hydrogen, and C₁-C₆alkyl.

6. The compound according to claim 1, wherein R³, R⁴ and R⁵ are hydrogen.

7. The compound according to claim 1, wherein A is selected from the group consisting of formula A-I to A-VII below 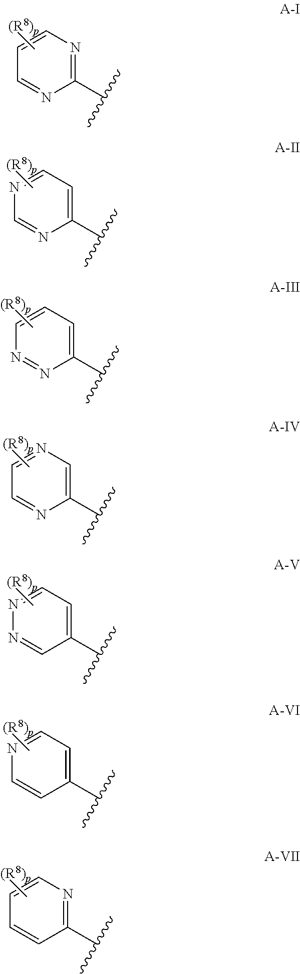
wherein the jagged line defines the point of attachment to the remaining part of a compound of Formula (I), p is 0, 1 or 2 and R⁸ is as defined in claim 1.

8. The compound according to claim 1, wherein A is selected from the group consisting of formula A-I to A-V below 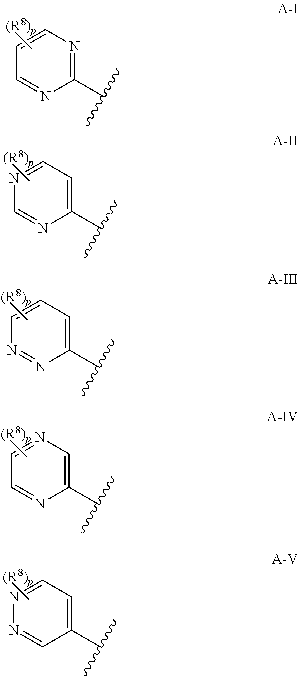
wherein the jagged line defines the point of attachment to the remaining part of a compound of Formula (I), p is 0, 1, or 2 and R⁸ is as defined in claim 1.

9. The compound according to claim 1, wherein when A is substituted by 1 or 2 substituents, each R⁸ is independently selected from the group consisting of halogen, nitro, cyano, —NH₂, —NHR⁷, —N(R⁷)₂, —OH, —OR⁷, —S(O)ᵣR¹⁵, —NR⁶S(O)₂R¹⁵, —C(O)OR¹⁰, —C(O)R¹⁵, —C(O)NR¹⁶R¹⁷, —S(O)₂NR¹⁶R¹⁷, C₁-C₆alkyl and C₁-C₆haloalkyl.

10. The compound according to claim 1, wherein when A is substituted by 1 or 2 substituents, each R⁸ is independently selected from the group consisting of chloro, fluoro, cyano, —NH₂, —N(Me)₂, —OMe, —S(O)₂Me, —C(O)NHMe, —C(O)N(Me)₂, methyl and trifluoromethyl.

11. The compound according to claim 1, wherein A is selected from the group consisting of formula A-I to A-V and p is 0.

12. The compound according to claim 1, wherein the terminal cyclic moiety of Z (i) is substituted by —OH, and/or (ii) comprises a ring nitrogen bearing a hydrogen atom.

13. The compound according to claim 1, wherein the terminal cyclic moiety of Z is linked to the rest of the compound of Formula (I) via a moiety comprising an —NH— group adjacent to an oxygen containing group, said oxygen containing group selected from the group consisting of —C(O), —S(O), and —S(O)₂.

14. The compound according to claim 1, wherein the terminal cyclic moiety of Z is selected from the group consisting of:
an optionally substituted 4-6 membered carbocyclic 1,3-dione;
an 5- or 6-membered heterocyclic ring comprising up to 4 heteroatoms independently selected from O, S, and N, substituted either on at least one ring carbon by —OH or an a ring nitrogen by hydrogen, and optionally substituted on any other ring atom; and,
an optionally substituted 5- or 6-membered heteroaromatic ring comprising at least one nitrogen atom and from 0 to 2 further heteroatoms independently selected from O, N and S.

15. The compound according to claim 1, wherein Z is selected from the group consisting of Z1 to Z69 as shown below: 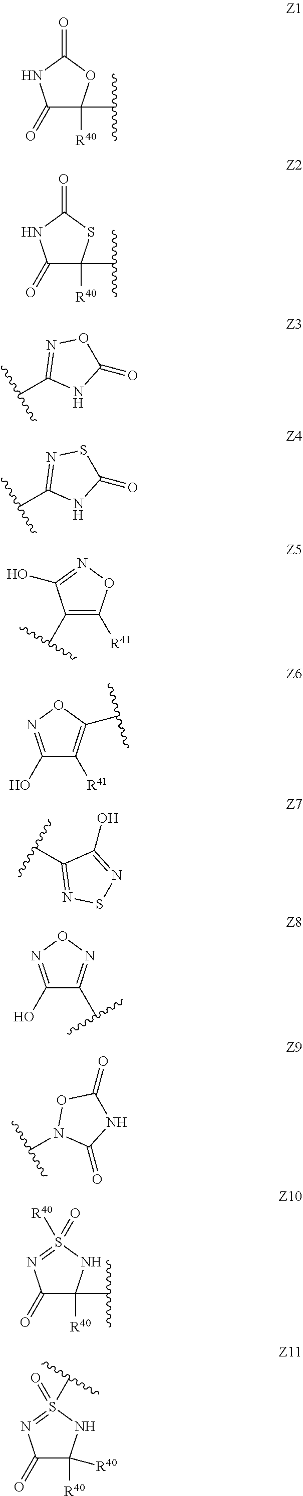 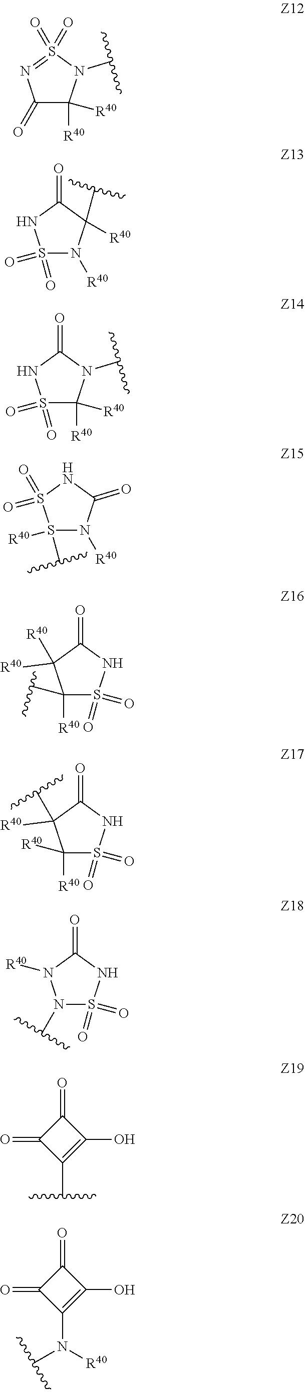 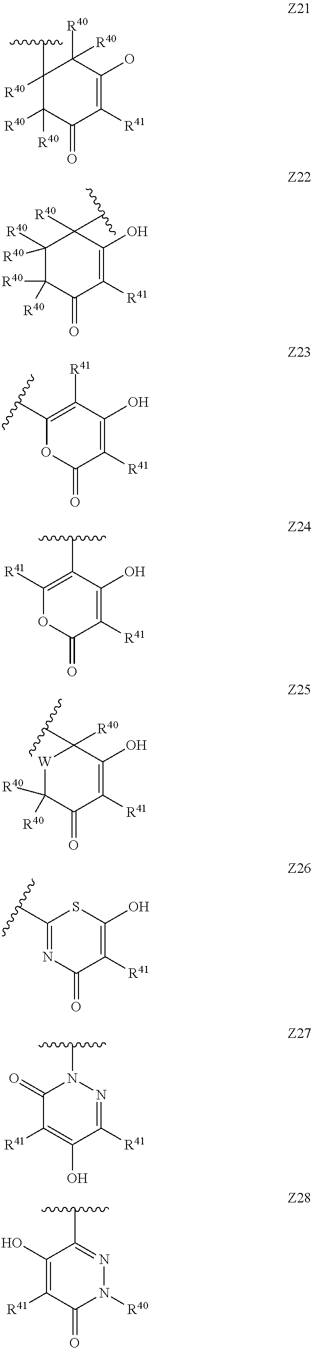 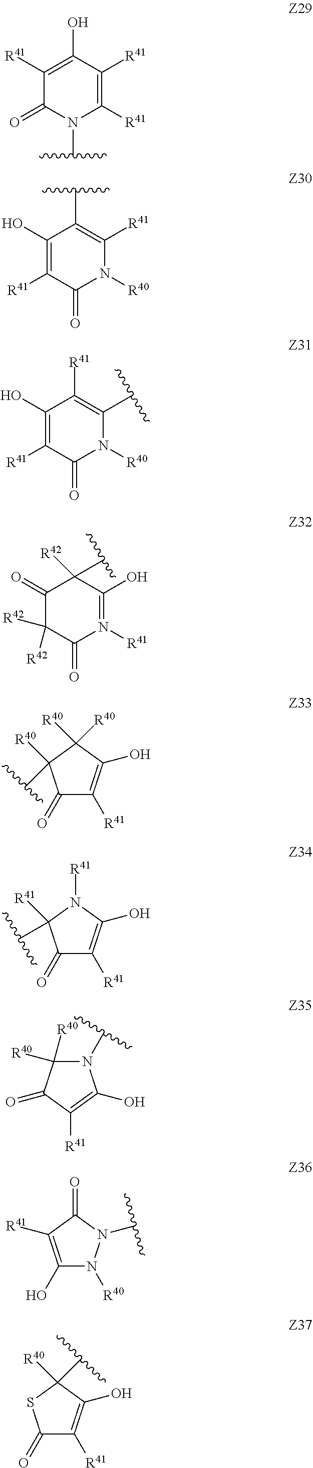 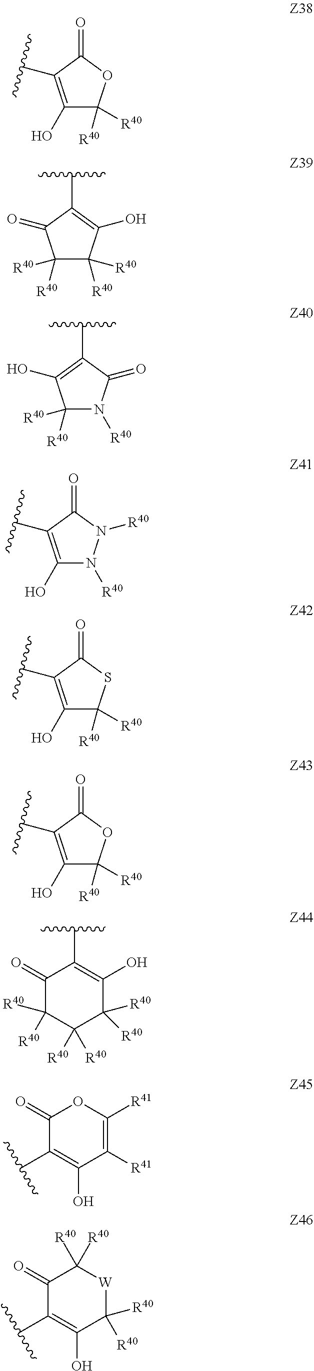 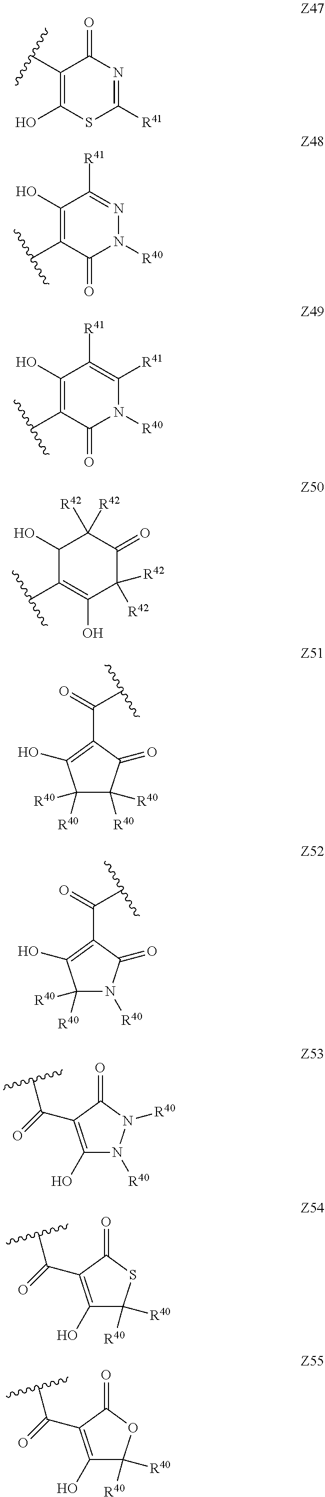 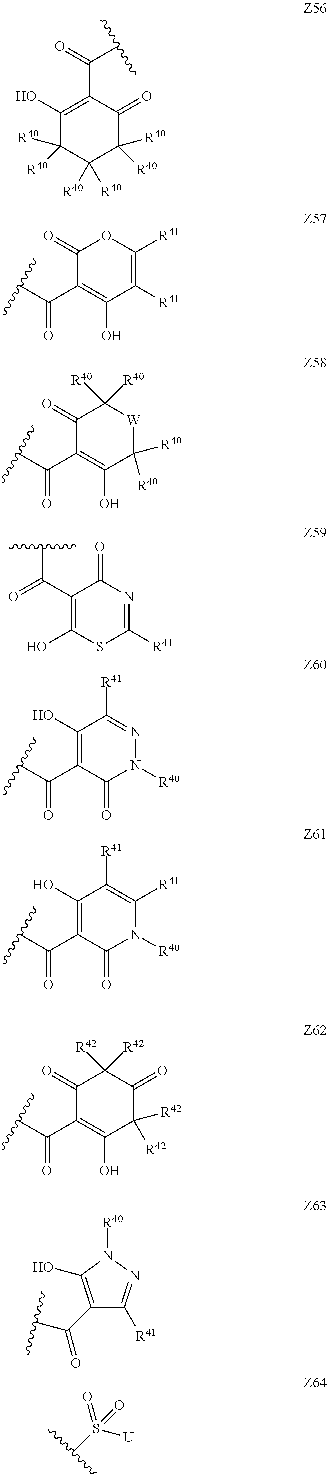 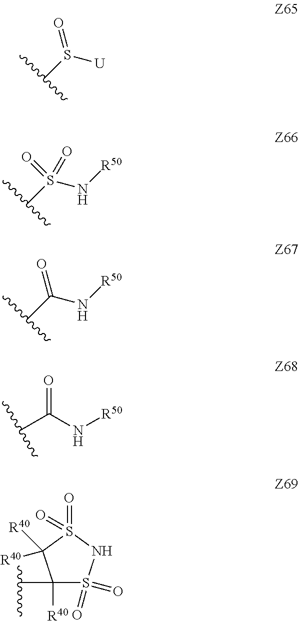
and wherein
each R⁴⁰ is independently selected from the group consisting of hydrogen, C₁-C₆alkyl, C₁-C₆alkoxy, C₁-C₃alkoxyC₁-C₃alkyl;
each R⁴¹ is independently selected from the group consisting of hydrogen, C₁-C₆alkyl, and C₁-C₃alkoxyC₁-C₃alkyl;
each R⁴² is independently selected from the group consisting of C₁-C₆alkyl and C₁-C₃alkoxyC₁-C₃alkyl;
W is selected from the group consisting of —O—, —S—, —S(O)—, —S(O)₂—, and —N(R⁴⁵)—;
R⁴⁵ is selected from the group consisting of hydrogen, C₁-C₃alkyl, C₁-C₃alkoxy, C₁-C₃alkoxyC₁-C₃alkyl, and C₁-C₆alkylcarbonyl;
U is a 5- or 6-membered heteroaromatic ring comprising at least one ring nitrogen atom substituted by hydrogen, and from 0 to 2 further heteroatoms independently selected from O, N and S, wherein said heteroaromatic ring is optionally substituted by 1, 2, or 3 R⁹ substituents;
R⁵⁰ is a 5- or 6-membered heteroaromatic ring comprising at least one ring nitrogen atom and from 0 to 3 further heteroatoms independently selected from O, N and S, wherein said heteroaromatic ring is optionally substituted by 1, 2, or 3 R⁹ substituents, and the jagged line denotes the point of attachment to the rest of the molecule.

16. The compound according to claim 1, wherein n is 0.

17. An agrochemical composition comprising a herbicidally effective amount of a compound of Formula (I) as defined in claim 1 and an agrochemically-acceptable diluent or carrier.

18. A method of controlling unwanted plant growth, comprising applying a compound of Formula (I) as defined in claim 1.

19. Use of a compound of Formula (I) as defined in claim 1, as a herbicide.

20. A herbicidal composition according to claim 17, to the unwanted plants or to the locus thereof.
